(19) United States
(12) Patent Application Publication
(10) Pub. No.: US 2026/0294293 A1
(43) Pub. Date: Oct. 1, 2026

(54) A PROGRAMMABLE, BATTERY-LESS, AUTONOMOUS AND BIODEGRADABLE SMART DUST PARTICLE

(71) Applicant: TECHNION RESEARCH & DEVELOPMENT FOUNDATION LIMITED, Haifa (IL)

(72) Inventor: Hossam HAICK, Haifa (IL)

(21) Appl. No.: 19/478,065

(22) PCT Filed: Apr. 21, 2024
(86) PCT No.: PCT/IL2024/050397

§ 371 Date: Oct. 23, 2025

Related U.S. Application Data
(60) Provisional application No. 63/463,966, filed on May 4, 2023.
(60) Provisional application No. 63/609,909, filed on Dec. 14, 2023.

(51) Int. Cl. A61B 5/1477; A61B 5/00; A61B 5/145; A61B 10/00; A61B 90/96; G01N 30/02; G01N 30/62; G01N 30/86

(52) U.S. Cl. CPC A61B 5/1477; A61B 5/0004; A61B 5/14546; A61B 5/6887; A61B 5/7246; A61B 10/0041; A61B 90/96; G01N 30/62; G01N 30/8624; A61B 2562/0285; A61B 2562/046; G01N 2030/025

(57) ABSTRACT

A system and method provide non-invasive, continuous monitoring using a plurality (swarm) of programmable, battery-less, autonomous, biodegradable smart dust particles (SDPs). Each SDP features sensors printed on a layered separating medium for chromatographic separation and an RFID tag comprising a unique identification (ID) barcode. Applied to skin overlying biological tissue, optionally via spray, the SDPs transmit signals wirelessly to an external receiver. A processor correlates received signals with spatial coordinates on the skin using each SDP's unique ID. This enables the generation of a spatiotemporal chemical tomogram, mapping detected chemical compounds (e.g., VOCs) across the underlying biological tissue. The system facilitates applications such as wearable tomography and continuous tumour diagnostics, including monitoring for conditions like breast cancer by analysing the chemical map over time.

### TECHNICAL FIELD

The present application relates to a programmable, battery-less, autonomous and biodegradable smart dust particle, system comprising the same, and a method for tumour diagnostics using the smart dust particles.

### BACKGROUND

While chemicals are essential to many aspects of our lives and provide key solutions, they can also pose environmental and health problems. Examples include air pollution, environmental change, technological hazards, biological weapons, and ever-increasing chronic diseases such as cancer, diabetes, neurodegenerative diseases, and more. Each of these problems involves a cocktail of hundreds or even thousands of chemicals, only a few of which have been identified.

This reality becomes even more apparent when considering that: (a) chemicals contribute to a target measurement (e.g., toxicity or biomarker detection) in a complex mixture, even if their presence is below their own exposure threshold and/or analytical detection limit; (b) chemicals with similar properties tend to follow the concept of “complementary action” in a mixture, whereas chemicals with different mechanisms of action act according to “independent action”; and (c) the spatial distribution of chemicals within a given object or between objects is far from uniform, even those that may be considered identical at the macroscopic level. Awareness of these complex chemical mixtures has increased since the advent of the exposome concept, which showed, among other things, that up to two-thirds of human chronic disease risk and 16% of premature deaths worldwide cannot be explained by genetics alone and may result from the environment or gene-environment interactions.

To maintain the benefits of chemicals in our lives, three basic principles must be adopted so that appropriate treatment can be carried out, mainly before disaster occurs: (1) precautions; (2) prevention and elimination of (unwanted) chemical compounds at source; and (3) dynamic monitoring of all stages of the life cycle of chemical compounds. The precautionary principle is a risk assessment tool that can be used when a risk to human health/environment is suspected. Such instruments should be non-discriminatory and proportionate and should provide measures and/or data that need to be revised as additional scientific information becomes available. On the other hand, since restoration of the natural environment is a very slow process, a continuous monitoring system is needed to provide early information about possible threats before hazardous compounds accidentally enter the human or animal body.

The present invention is directed at a conceptually new approach to provide detailed information on a wide range of chemicals and their mixtures through a wireless network of analytical and/or sensor instruments that can exist anywhere, spanning local and global scales, in real time, and depending on spatial location and associated variability (for example, accidental releases of biochemicals and harmful compounds, pesticides in agriculture, sudden outbreaks of biological weapons, etc.) Of particular importance in each scenario is the need not only for target identification in space and time, but also for non-target identification, the latter being an important predictor of the occurrence of new types of disasters.

### SUMMARY

The present invention relates to a programmable, battery-less, autonomous and biodegradable smart dust particle (SDP) comprising:
A. a single layer or plurality of layers of a layered separating media for chromatographic separation of compounds contained in a sample subjected to the separation and for providing physical support for embedded electronics and for at least one detector;
B. said at least one detector comprising an array of sensors printed on the layers of said separating media, and providing information on the presence and properties of said compounds in the sample;
C. said embedded electronics printed on the layers of said separating media; and
D. at least one radio-frequency identification (RFID) out-input tag for remote readout and zero-power operation, each RFID tag connected to said embedded electronics via an electric circuit for receiving or transmitting a signal.

In one embodiment, the SDP of the invention further comprises environmental sensing layers, light sensing layers, mass sensing layers, sound sensing layers, and other layers comprising a variety of physical and mechanical sensors. The environmental sensing layers may comprise temperature, humidity and pressure sensors. The sensing layers are printed on said layered separating media using graphene or carbon-nanotube ink solution, or any other nanomaterials that either conductive or semi-conductive, and natural silk protein solution. The sensors are screen-printed or inkjet-printed on each layer of said separating media.

In another embodiment, the detector of the SDP is selected from a micro-gas chromatograph, miniaturised dispersive optical spectrometer, fibre-coupled optical spectrometer, MEMS-based spectrometer, plasmon-enhanced Raman spectrometer, on-chip plasmonic spectrometer, piezoelectric crystal detector, and spin-induced mass spectrometer, or combinations thereof. The detector may further comprise one or more microfabricated components. The microfabricated components are selected from capillary or chip-based microcapillary separation columns, a source of carrier gas, pre-concentrator-injector, micro-and/or nano-optical components, microfluidics components, pumps, filters, and valves. The detector further comprises hardware and software for instrument control, data acquisition, and analysis.

In a further embodiment, sensors of the SDP are selected from:
(a) thermal conductivity sensors designed to detect the difference in thermal conductivity between the sample and a stream of pure carrier gas passed through a reference cell;
(b) surface acoustic wave (SAW) sensors designed to detect changes in propagation characteristics of acoustic waves near the surface of a piezoelectric material;
(c) chemiresistor array sensors comprising gold-thiolate monolayer-protected nanoclusters (MPNs) deposited onto patterned microelectrodes, and designed to detect changes in resistance as a result of absorption of vapor molecules (chemical vapours) by the nanoclusters;
(d) chemicapacitive array sensors comprising two parallel electrodes or interdigitated electrodes, a sensing material, or a polymer film, either sandwiched between the pair of parallel electrodes or coated on the interdigitated electrode, and designed to detect changes in the capacitance of the sensor as a result of absorption of chemical vapours leading to a swelling of polymer and change in electrical permittivity, as a function of the tested compounds concentration; and
(e) nanocantilever sensors comprising a beam resonator structure coated with a sensing material, or a polymer, that is supported on a rigid support, and designed to detect changes in the resonance frequency as a result of absorption of chemical vapours, proportional to the amount of mass absorbed.

The sensors of the SDP may further comprise at least one chemical or biomolecular layer immobilised on top of said sensors and capable of binding or adsorbing said compounds from the sample. Said at least one chemical layer comprises chemical functional groups selected from amines, alkenes, alkynes, phosphines, azides, cycloalkenes, cycloalkynes, cyclopropanes, isonitriles, vinyl boronic acid, tetrazine, maleimide, alcohols, thiols, conjugated dienes, copper acetylide, nitrones, aldehydes, ketones, alkoxyamines, hydroxylamine, hydrazine, hydrazide, isothiocyanate, carbodiimide, and carboxylic acids or derivative thereof, esters, anhydrides, N-hydrosuccinimide (NHS), tosyl and acyl halides.

Said at least one chemical or biomolecular layer comprises cyclodextrin, 2,2,3,3-tetrafluoropropyloxy-substituted phthalocyanine or derivatives thereof, or said chemical or biomolecular layer comprises capturing biological molecules, primary, secondary antibodies or fragments thereof against certain proteins to be detected, or their corresponding antigens, enzymes or their substrates, short peptides, specific polynucleotide sequences, which are complimentary to the sequences of DNA to be detected, aptamers, receptor proteins or molecularly imprinted polymers.

In yet further embodiment, selectivity of the sensors is altered by changing the chemical identity of said at least one chemical or biomolecular layer.

In still another embodiment, the compounds contained in the sample subjected to the separation are selected from:
hydrocarbons;
alcohols;
enantiomers, spatial and structural isomers of organic compounds;

industrial solvents;
fuel oxygenates;
by-products produced by chlorination in water treatment;
petroleum fuels, hydraulic fluids, paint thinners, and dry-cleaning agents;
common ground-water contaminants;
isoprene, terpenes, pinene isomers and sesquiterpenes;
regulated ozone-depleting chlorinated hydrocarbons;
food toxins, aflatoxin, shellfish poisoning toxins, saxitoxin or microcystin;
neurotoxic compounds, methanol, manganese glutamate, nitrix oxide, tetanus toxin or tetrodotoxin, Botox, oxybenzone, Bisphenol A, or butylated hydroxyanisole;
explosives, picrates, nitrates, trinitro derivatives, 2,4,6-trinitrotoluene (TNT), 1,3,5-trinitro-1,3,5-triazinane (RDX), trinitroglycerine, N-methyl-N-(2,4,6-trinitrophenyl)nitramide (nitramine or tetryl), pentaerythritol tetranitrate (PETN), nitric ester, azide, derivates of chloric and perchloric acids, fulminate, acetylide, and nitrogen rich compounds, tetrazene, octahydro-1,3,5,7-tetranitro-1,3,5,7-tetrazocine (HMX), peroxide, triacetone trioxide, C4 plastic explosive and ozonidesor, or an associated compound of said explosives, decomposition gases or taggants, and
biological pathogens, a respiratory viral or bacterial pathogen, an airborne pathogen, a plant pathogen, a pathogen from infected animals or a human viral pathogen.

In additional embodiment, a system for performing an autonomous analysis of a sample taken from an environment, said sample containing compounds subjected to a chromatographic separation, said analysis is performed by processing spectrometry data of the sample, said system comprises:
(a) a plurality of the SDPs of the present invention forming a smart dust;
(b) external controlling and monitoring means for controlling and monitoring the SDPs by an operator; and
(c) an external memory.

In yet further embodiment, a system for tumour diagnostics comprises:
(a) a plurality of the SDPs of any one of claims 1 to 15 forming a smart dust suitable for applying said SDPs as a thin film onto skin of a patient or to a skin ex vivo sample taken from the patient, and for transmitting spectrometry data received from said SDPs to an external controlling and monitoring means;
(b) said external controlling and monitoring means for controlling and monitoring the SDPs by an operator; and
(c) an external memory.

Both systems of the present invention may further comprise a wireless connection module for connecting said SDPs to said external memory. The external memory may comprise another wireless connection module connecting said system to an operator's interface via a digital-to-analogue converter (DAC).

In some embodiments, communication between the SDPs and the external memory is either passive or active, or combination thereof. If the communication between the SDPs and the external memory is passive, the system is then configured to perform a spectral encoding of information using a single radiative structure with multiple resonators each of which is dedicated either to a bit encoding or to a sensor readout. If the communication between the SDPs and the external memory is active, the system is then configured to carry out a parallel route for powering and communicating between the sensors and external memory using a semiconductor device.

In a particular embodiment, the external memory is selected from a mobile device, wearable gadget, smartphone, smartwatch, smart ring, desktop computer, server, remote storage, internet storage, and internet cloud. The external memory comprises a processor, a microcontroller or a memory-storing controller suitable for storing executable instructions, which when executed by the processor cause the processor to perform a machine-learning method on the measurement results.

In a certain embodiment, both systems of the present invention further comprises a remote powering with miniaturised receiver antenna.

In another aspect of the present invention, a method for tumour diagnostics comprises:
Applying the SDPs of the present invention as a thin film onto skin of a patient or to a skin ex vivo sample taken from the patient;
Transmitting spectrometry data received from said SDPs to a signal receiver;
Processing said data by Al; and
Performing an autonomous analysis to output the result as a heatmap defined as a chemical tomogram.

In the method of the present invention, the SDPs are optionally applied to the patient's skin in a form of a spray above premalignant and tumorous tissues. Signals from said SDPs are then sampled and analysed through the RFID out-input tags. Biomarkers identified by said SDPs are detected through the patient's skin.

In a specific embodiment, the Al analysis is performed in the method of the present invention to define biomarker combinations. Said diagnosed tumours and biomarkers may be from different types of cancer. In another specific embodiment, said diagnosed tumours and biomarkers are from pre-malignant breast lesions, breast cancer cells and tumour microenvironments, cultured human cell lines from premalignant breast lesions, breast cancer cells that represent the different molecular subtypes of breast cancer, stroma, extracellular matrix, immune cells, and vasculature.

According to the embodiments of the present invention, the system of the invention is used in tomography and tumour diagnostics.

In a further aspect of the present invention, a wearable tomography device for lifelong non-invasive and continuous monitoring and molecular imaging of cancer comprises at least one SDP of the present invention, or connected to a system of the present invention.

Various embodiments may allow various benefits and may be used in conjunction with various applications. The details of one or more embodiments are set forth in the accompanying figures and the description below. Other features, objects and advantages of the described techniques will be apparent from the description and drawings and from the claims

### BRIEF DESCRIPTION OF THE DRAWINGS

Disclosed embodiments will be understood and appreciated more fully from the following detailed description taken in conjunction with the appended figures. The drawings included and described herein are schematic and are not limiting the scope of the disclosure. It is also noted that in the drawings, the size of some elements may be exaggerated and, therefore, not drawn to scale for illustrative purposes. The dimensions and the relative dimensions do not necessarily correspond to actual reductions to practice of the disclosure.

FIG. 1A shows the state-of-the-art, one-time sampling from the confined and small area, and chemical analysis for complex chemical mixtures, characterised by the passive sampling procedure, minimal cleanup, extensive target analysis and suspect and non-target screening, limited in space and time.

FIG. 1B shows the sampling through time from unlimited area in the present invention, and chemical analysis for complex chemical mixtures, characterised by the autonomous and continuous sampling procedure with RF-connected SDPs, minimal to none cleanup, target and non-target analysis, and mapping spectrum of compounds everywhere and over time. The spotlight in FIG. 1A and FIG. 1B demonstrates the spectrum of compounds that can be detected in each approach.

FIGS. 2A-2B schematically demonstrate the overall design concept of the present invention. FIG. 2A shows the overall architecture of the designated programmable, battery-less and biodegradable smart dust particle (SDP) of the present invention, which is also called a biodegradable mass-spectrometry-in-a-particle (BMSP). FIG. 2B shows an expanded view on the main sensing layers (with uGC) and related components therein and signals obtained from each individual chemical compound received from each layer upon exposure to a complex mixture. The top image in FIG. 2C schematically shows the SDP swarms in the environment using RF for energy-harvesting and communication between various SDPs and between the SDPs and external receiver; and the bottom image shows biodegradability of these SDPs with time at the end of their activity.

FIGS. 3A-3G show the schematic layout of the various steps in the fabrication process of the SDPs. FIG. 3A schematically shows the printed VOC sensors (1), temperature sensor (2) and humidity sensor (3), and FIG. 3B illustrates the printed electrodes (4) and antennas (5). FIG. 3C and FIG. 3D schematically show the multitask arrangement for each printed layer in hierarchical arrangement. FIG. 3E schematically shows the cross-section of the SDP with a protection layer, FIG. 3F illustrates the 3D-printed protection layer encasing the SDP unit, and FIG. 3G shows the separation of the VOC components V₁, V₂ . . . Vₙ in the mixture based on their molecular weight (M. W.) with the SDP of the present invention.

FIG. 4A schematically shows the design of the mass scale printed sensor and antenna electrode on a sheet paper, FIG. 4B illustrates the layered arrangement of each layer, and FIG. 4C shows this design with the top and bottom porous layers covering the separation layers. FIG. 4D demonstrates the 3D printing of the protection layers on a mass scale, and FIG. 4E illustrates the dicing of an individual dust sensor.

FIG. 5 schematically shows the communication architecture with one shown cluster or with multiple clusters, where (10) is a mini-gateway, (11) is an access point (Wi-Fi or cellular), (12) is Internet (server or cloud), (13) is apps and data visualisation, and d is a distance of hundreds of metres up to several kilometres (depends on the communication technology used, for instance GSM, GPS or similar).

FIG. 6A schematically shows the experimental arrangement for the SDP devices (100) for the gas (VOC) mixture (101) sensing inside the simulation chamber (102) using multiband and single frequency measurements scheme when the single SDP units are kept at a longer distance.

FIG. 6B schematically shows the experimental arrangement for the SDP devices for the gas (VOC) mixture (101) sensing inside the simulation chamber using multiband and single frequency measurements scheme when the multiple SDP units are placed very close to each other, and the concentration mapping during the elution of the mixture from the simulation chamber.

FIG. 6C schematically shows the principle of the operation of the SDP devices, where an RF source represents a spherical waveform reaching all the SDPs simultaneously. Consequently, a frequency tuning as shown in FIG. 6D must be done to take the data wirelessly.

FIGS. 7A-7E schematically show the entire printed system of the present invention comprising the RF powered mass spectrometry on the all-printed smart dust sensor in the folded layer-by-layer design, and it is sealed. FIG. 7A shows the system in the unfolded state, where 110 is an inkjet-printed sensor, 111 is an inkjet-printed electrode, 112 are folding lines, 113 is a microprocessor, 114 is a transmitting antenna, 115 is a printed filter, 116 is a printed rectifier, 117 is a printed radio-frequency (RF) receiving antenna, 118 is a temperature sensor, and 119 is a humidity sensor. FIG. 7B shows the system being origami-folded along the folding lines 112, and FIG. 7C shows the semi-folded system after folding the layers. FIG. 7D shows the semi-folded system with the top porous layer 120, and FIG. 7E shows the completely folded and sealed system of the present invention.

FIG. 8A schematically shows the SDP structure of backscattering antenna in a passive implementation with sensors tuned resonators, where 200 are the folding lines, 201 is a connecting wire to sensors, 202 is a VOC sensor, 203 is a temperature sensor, and 204 is a humidity sensor.

FIG. 8B schematically shows the SDP structure in an active implementation with the Si-based energy harvesting, sensor redout and communication, where 205 is a sensors' ground and antenna, 206 is a silicon DIE (25 μm), which decouples the sensing from the communication, and 207 is a folding gap, and FIG. 8C shows the stacking of the layers, followed by creating the smart dust particle 208 (SDP or SDP) of the present invention.

FIG. 9 schematically shows the overall operational diagram and chart including the communication scheme dedicated to sensing swarm data gathering and consolidation.

FIG. 10 shows a design concept of the method of the present invention for tumour diagnostics.

FIGS. 11A-11N show the bridging multi-omics data with the Al-enabled SDP device. FIG. 11A shows the AI-enabled SDP devices of the present invention that elucidate the spatiotemporal separation of VOCs based on molecular weight from the organoid samples' environment. FIG. 11B shows the SDPs map of various eluted volatile organic compounds (VOCs) from the organoid using frequency domain spectrogram, and FIG. 11C shows the 2D chemical tomography through sensor fusion.

FIG. 11D schematically represents the bridging volatolomics, which is the study of VOCs emitted by a biological system, under specific experimental conditions, with multi-dimensional imaging as well as proteo-genomics using generative AI.

FIG. 11E shows a stepwise scheme demonstrating breast cancer progression (normal (MCF10A (M1)), premalignant (MCF10AT (M2)) and malignant (MCF10CA1h (M3)) breast cells),

FIGS. 11F-11G show the DAPI staining characterization of the progression of breast cancer, and FIGS. 11I-11K shows the microscope images of this process (with magnification ×40, Bar=50 μm).

FIG. 11L shows the western blot (WB) for the expression of mesenchymal markers (i) fibronectin, (ii) vimentin), (iii) E-cadherin (epithelial marker) with a (iv) simple protein loading (tubulin). FIG. 11M and FIG. 11N shows the quantification of fibronectin and vimentin levels, respectively, from WB results. Densitometry values were normalized to M1. Columns; mean, bars; SD, n=3. * P<0.05, ** P<0.01, *** P<0.001.

FIG. 12A shows the spectrometer circuit module (301) used for extracting VOCs from organoids with the SDP of the present invention. The circuit and block diagram of the spectrometer module to interface the SDP's layers L1, L2 and L3, as seen in the figure, with I²C communication (302) between Slaves 1, 2 and 3, communication port (303), Master (304), display unit (305), temperature, flow, switching and other controllers (306) and data acquisition and storage system (307).

FIGS. 12B-12D shows the layer-dependent sensing using the SDP of the present invention and their comparative spectrum analyses for M1-M3 and the respective background (media) for Layer 1 (FIG. 12B), Layer 2 (FIG. 12C) and Layer 3 (FIG. 12D) using 1-4-phenylene-diamine-based sensing probe. FIGS. 12E-12G shows the corresponding spectrograms for Layers 1-3, respectively. FIGS. 12H-12J shows the influence of sensor chemistries at Layer 2 on scalogram obtained from the SDP's spectrogram during transition from M1-M3 for other typical ligands: L-cysteine (amine) (FIGS. 12H), 2-napthalene thiol (thiol) (FIGS. 12I) and 2-amino-4-chlorobenzene-thiol (amine and thiol) (FIG. 12J).

FIGS. 12K-12P shows the heatmaps from gaussian fitting of splitting sensing profile in Layer 2 (FIGS. 12K-12M) and Layer 3 (FIGS. 12N-12P) for M1-M3, respectively, measured with 20 different ligands (X axis) for each case. FIG. 12Q shows the GC-MS heatmap analyses for each of 36 VOC samples (VOC1-VOC36). The values on the right-hand side of the scale are represented in logarithmic units.

FIGS. 13A-13C show the compound image processing inspired from ‘ommatidium’ mechanism in ‘bee vision’ to predict organoid's heterogeneity. FIG. 13A illustrates the concept of the invention which is creating hybrid compound mosaic images (2D tomography) by amalgamating sensor chemistries within distinct layers. These images are then harnessed in deep neural network image processing to predict the specific organoid's image pixel by pixel.

FIG. 13B schematically shows generative deep learning models for organoid image synthesis using an encoder-decoder based U-net architectures tailored for generating organoid images, both stained and unstained.

FIG. 13C schematically shows the sensing profile from 20 sensors/layers. The utilization of 20 sensor inputs yields synthetic superpositions for each layer (Layer 2 and 3), resulting in distinctive mosaic arrangements.

Reference is now made to FIGS. 14A-14B showing the generative deep learning models for organoid image synthesis. The resulting AI-generated images, representing various training cycles that demonstrate normal microscope images (transformed from grayscale to false colour) are shown in FIG. 14A, and DAPI-stained images for M1, M2, and M3 shown in FIG. 14B, respectively, for each case. Each image's training cycle number is indicated in the right-hand side corner at the bottom of the image.

FIGS. 15A-15D shows Genomescope-AI, Sequencescope-AI and Proteoscope-AI for volatolomics-proteomics-genomics fusion and genetic prediction of organoids.

FIG. 15A schematically shows the AI architecture that explores the VOC-genetic correlation, and FIG. 15B shows the Genomescope-AI predicted DNA copy number variation (CNV) at various cytoband position of target chromosome for M1, M2, M3 in the form of heatmap (Yes/No) for gain and loss. Each case is depicted with distinct colour and the number of iterations with corresponding predicted heatmap output is shown in the top of each case.

FIG. 15C shows the Sequencescope-Al predicted RNA sequences of (i)-(iii) M1-M3 and their mutation position, and FIG. 15D shows the Proteoscope-Al predicted WB results for various protein expressions, such as (i) vimentin, (ii) fibronectin, (iii) E-Cadherin and (iv) tubulin (loading control) for M1-M3.

FIG. 15E shows the generated 3D rendition for vimentin WB analyses indicating the increase of height and area which can be used for quantitative predictions.

### DETAILED DESCRIPTION

In the following description, various aspects of the present application will be described. For purposes of explanation, specific configurations and details are set forth in order to provide a thorough understanding of the present application. However, it will also be apparent to one skilled in the art that the present application may be practiced without the specific details presented herein. Furthermore, well-known features may be omitted or simplified in order not to obscure the present application.

The term “comprising”, used in the claims, is “open ended” and means the elements recited, or their equivalent in structure or function, plus any other element or elements which are not recited. It should not be interpreted as being restricted to the means listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a device comprising x and z” should not be limited to devices consisting only of components x and z. Also, the scope of the expression “a method comprising the steps x and z” should not be limited to methods consisting only of these steps.

Unless specifically stated, as used herein, the terms “about” and “approximately” are understood as within a range of normal tolerance in the art, for example within two standard deviations of the mean. In one embodiment, the term “about” means within 10% of the reported numerical value of the number with which it is being used, preferably within 5% of the reported numerical value. For example, the term “about” can be immediately understood as within 10%, 9%, 8%, 7%, 6%, 5%, 4%, 3%, 2%, 1%, 0.5%, 0.1%, 0.05%, or 0.01% of the stated value. In other embodiments, the term “about” can mean a higher tolerance of variation depending on for instance the experimental technique used. Said variations of a specified value are understood by the skilled person and are within the context of the present invention. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges, for example from 1-3, from 2-4, and from 3-5, as well as 1, 2, 3, 4, 5, or 6, individually. This same principle applies to ranges reciting only one numerical value as a minimum or a maximum. Unless otherwise clear from context, all numerical values provided herein are modified by the term “about”. Other similar terms, such as “substantially”, “generally”, “up to” and the like are to be construed as modifying a term or value such that it is not an absolute. Such terms will be defined by the circumstances and the terms that they modify as those terms are understood by those of skilled in the art. This includes, at very least, the degree of expected experimental error, technical error and instrumental error for a given experiment, technique or an instrument used to measure a value.

As used herein, the term “and/or” includes any and all combinations of one or more of the associated listed items. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached to”, “connected to”, “coupled with”, “contacting”, etc., another element, it can be directly on, attached to, connected to, coupled with or contacting the other element or intervening elements may also be present. In contrast, when an element is referred to as being, for example, “directly on”, “directly attached to”, “directly connected to”, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

The term “compound/s”, “tested compound/s” or “chemical compound/s” used in the present application are entirely equivalent and encompass virtually any chemical and biomolecular compound, including but not limited to organic compounds, volatile organic compounds (VOCs), biochemicals, etc. By common definition, “chemical compound is any substance composed of identical molecules (that can be biomolecules as well) consisting of atoms of two or more chemical elements”. Non-limiting examples of chemical compounds being tested in the present invention and then included in a library of micro-GC (uGC) patterns of the present invention are:
hydrocarbons;
alcohols;
enantiomers and structural isomers of organic compounds;
industrial solvents, such as trichloroethylene;
fuel oxygenates, such as methyl tert-butyl ether (MTBE);
by-products produced by chlorination in water treatment, such as chloroform;
petroleum fuels, hydraulic fluids, paint thinners, and dry-cleaning agents;
common ground-water contaminants;
isoprene, terpenes, pinene isomers and sesquiterpenes;
regulated ozone-depleting chlorinated hydrocarbons;
food toxins, such as aflatoxin, and shellfish poisoning toxins, such as saxitoxin or microcystin;
neurotoxic compounds, such as methanol, manganese glutamate, nitrix oxide, tetanus toxin or tetrodotoxin, Botox, oxybenzone, Bisphenol A, or butylated hydroxyanisole;
explosives, such as picrates, nitrates, trinitro derivatives, such as 2,4,6-trinitrotoluene (TNT), 1,3,5-trinitro-1,3,5-triazinane (RDX), trinitroglycerin, N-methyl-N-(2,4,6-trinitrophenyl)nitramide (nitramine or tetryl), pentaerythritol tetranitrate (PETN), nitric ester, azide, derivates of chloric and perchloric acids, fulminate, acetylide, and nitrogen rich compounds, such as tetrazene, octahydro-1,3,5,7-tetranitro-1,3,5,7-tetrazocine (HMX), peroxide, such as triacetone trioxide, C4 plastic explosive and ozonidesor, or an associated compound of said explosives, such as a decomposition gases or taggants, and
biological pathogens, such as a respiratory viral or bacterial pathogen, an airborne pathogen, a plant pathogen, a pathogen from infected animals or a human viral pathogen.

In one aspect of the present invention, a programmable, battery-less, autonomous and biodegradable smart dust particle (SDP) comprising:
i. a single layer or plurality of layers of a layered separating media for chromatographic separation of compounds contained in a sample subjected to the separation and for providing physical support for embedded electronics and for at least one detector;
ii. said at least one detector comprising an array of sensors printed on the layers of said separating media, and providing information on the presence and properties of said compounds in the sample;
iii. the embedded electronics printed on the layers of said separating media; and
iv. at least one radio-frequency identification (RFID) out-input tag for remote readout and zero-power operation, each RFID tag connected to said embedded electronics via an electric circuit for receiving or transmitting a signal.

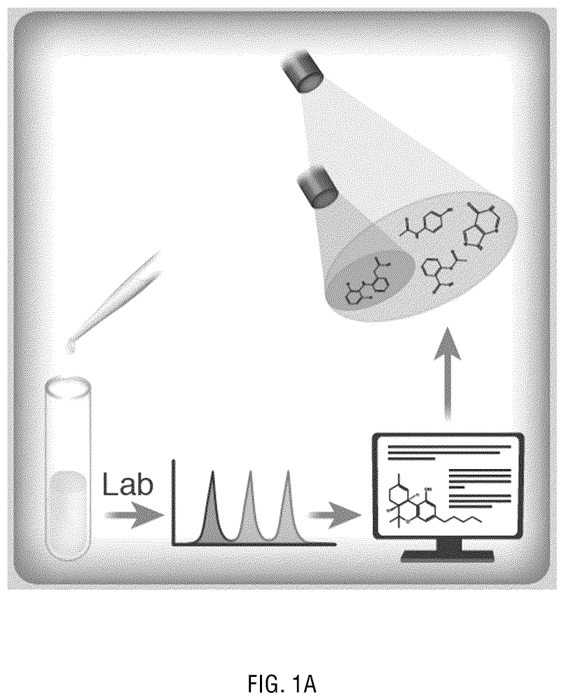

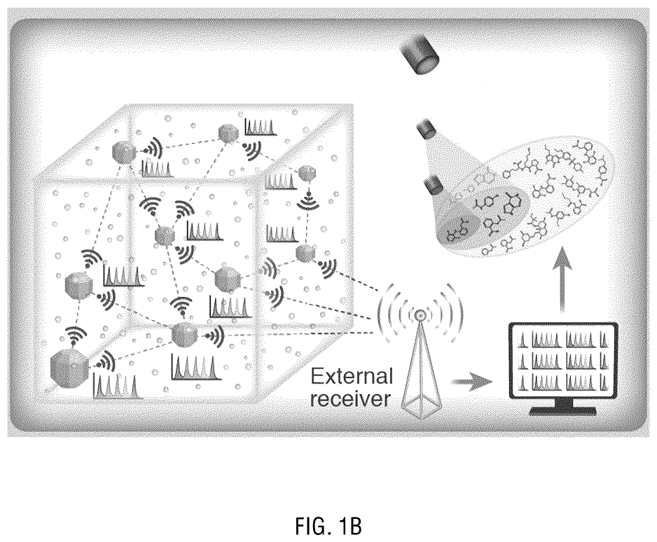

FIGS. 1A and 1B demonstrate the sampling and chemical analysis of complex mixtures via the state-of-the-art strategies as well as the developed concept of the present invention, respectively. FIG. 1A shows the state-of-the-art, one-time sampling from the confined and small area, and chemical analysis for complex chemical mixtures, characterised by the passive sampling procedure, minimal cleanup, extensive target analysis and suspect and non-target screening, limited in space and time. FIG. 1B shows the sampling through time from unlimited area in the present invention, and chemical analysis for complex chemical mixtures, characterised by the autonomous and continuous sampling procedure with RF-connected SDPs, minimal to none cleanup, target and non-target analysis, and mapping spectrum of compounds everywhere and over time. The spotlight in FIGS. 1A and 1B demonstrates the spectrum of compounds that can be detected in each approach.

As of today, the choice of sensing tool depends on the intended goal of analysis, the compound(s) being targeted and the setting mode. Of the variety of available tools, laboratory-based or even compact spectrometry are considered to be the most powerful. This is because, spectrometry offers comprehensive analysis, quantitation, and identification of a wide range of chemical compounds, and is highly suited to discovery studies of small inorganic and/or organic molecules.

Currently available spectrometry tools, often coupled with either gas or liquid chromatography, can detect tens of thousands of chemical compounds. They offer the possibility of performing routine target analysis, suspect screening, and discovery-based non-target analysis (NTA) in an all-in-one approach. Although target analysis remains an essential component of chemical risk assessment, this approach illuminates only a narrow portion of chemical exposures and offers no information on unknown or previously unexpected chemical compounds that fall outside the targeted method.

With NTA, the limited scope of priority chemical compounds is left behind, as even unknown masses can now be tracked in various applications, including performing retrospective screening of emerging contaminants (see FIG. 1A). NTA, especially coupled with suspect screening of classes of compounds, can complement targeted analytical techniques but can only supply part of the picture of the chemicals in complex samples. Even with the best NTA methods, including those combined with computational workflows that are based on exact mass and fragment matching, some chemicals remain outside the spotlight, such as those that elute too early or late from the column, are poorly ionized by existing ionization methods, or are not yet interpreted correctly with current knowledge. In each of the operation schemes, most spectrometry tools in use are still bulky and demand extensive sample pre-preparation and trained personnel, therefore, are not suitable for mapping mixtures to determine the distribution of chemicals across time, space, or various matrices.

Recent years have witnessed significant advances in scaling down spectrometry to handheld or on-chip portable sizes for attaining indicative, instantaneous and on-the-spot results, rather than transporting samples to a laboratory for analysis. Though these devices were demonstrated in wide variety of applications (e.g., soil and crop analysis, quality control of food products, and environmental scientific research), the reduction of their size has been associated with degradation of their resolution, dynamic range, or signal-to-noise ratio. Nevertheless, further miniaturization of spectrometry, down to the submillimetre scale, while maintaining high performances, could provide access to very important and unexplored opportunities in a wide range of applications. This includes, for example, in-situ or even in-vitro mapping of chemicals or disease biomarkers, correlation of spectral information with spatial data for revolutionizing large-scale monitoring, such as in crop or contaminations monitoring.

Recent studies have tried to use selective/lock-and-key sensors, which can be tuned to respond to specific substances, or cross-reactive arrays in conjugation with pattern recognition methods, which can be tuned to respond to specific substances and/or as chemical fingerprints of mixtures, as a way to overcome the challenges associated with spectrometry, mainly when continuous monitoring as a function or time and spatial coordination (i.e., chemical mapping) is required. Nevertheless, current sensing technologies provide superimposed signal(s) towards all compounds in the sample. For cross-reactive sensors in conjugation with machine learning methods, the countless possible combination of each component in a mixture, which need to be tested and trained for a reliable prediction in real sample analysis, complicates and decreases the reliable prediction of each individual compound in real sample analysis.

Several categories of sub-millimetre-scale autonomous sensing platforms, commonly attributed to “smart dust” platform, have been reported in various fields, including senso-transmitter, aerosolise electronics, wearable/implantable electronics, nano-bio-robotics, nano-machines, colloidal state machines, etc. These technologies have been platformed in cm to micrometre scale, depending on the application and fabrication procedure. Predominantly dominated by standard lithography and MEMS-based micro-machining, the devices are well equipped with single/multiple functionalities, such as temperature, pH, pressure. A recent report about ingestible electronic sensor showed that it can measure different gases (e.g., oxygen, hydrogen, and carbon dioxide) in the gut to distinguish changes in a person's diet and could potentially be used to help develop individualized diets. A colloidal nano-electronic state machines based on 2D materials for aerosolise electronics were demonstrated for detecting specific gases (e.g., triethylamine and ammonia) in simulated environment.

Despite showing a high prominence for reaching far-accessing regions where traditional detectors are hard to implement, it still remains challenging for these chemically-sensitive miniaturized sensors to detect compounds that were not targeted during the design and fabrication process of the sensors and/or to reliably detect and distinguish between multiple gas compounds in a similar fashion to that of the spectrometry. In an era of uncertainty, with COVID-19 pandemics being one of the most prominent examples, this limitation would lead to mistaken evaluations and, therefore, improper management of an event or catastrophe. An approach that involves simply scaling down benchtop spectrometers and inter-communication between them becomes constrained because of the complex fabrication and the inherent resolution for separating specific compounds from the mixture cocktail in dispersion-based systems. On top of these challenges, wide-scale adoption of these technologies would bring with it several risks: (i) Control—Once billions of sub-millimetre sensing devices are deployed over an area it would be difficult to retrieve or capture them if necessary, making it challenging for the authorities to control. (ii) Pollution—Sub-millimetre sensing devices are essentially single-use devices. Unless they are fully biodegradable the question arises if they will pollute the areas where they are used (soil, air, water). (iii) Health—As soon as sub-millimetre sensing devices shrink to the sub-millimetre or nanoscale regimes; they will have a health risk when they are inhaled or ingested. (iv) Cost—As with any new technology, the cost to implement sub-millimetre sensing devices, which includes the satellites and other elements required for full implementation, is high. Until costs come down, it will be technology out of reach for many.

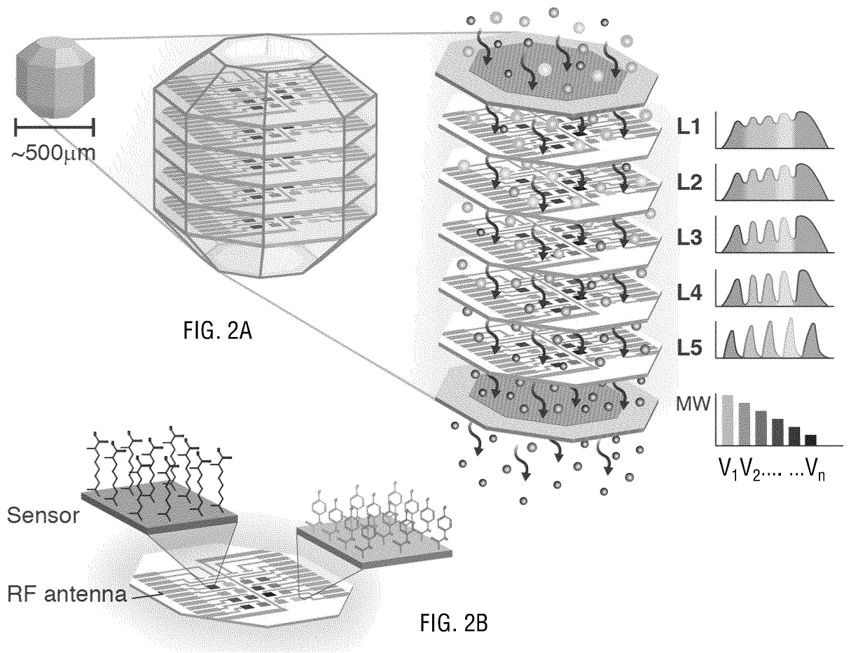

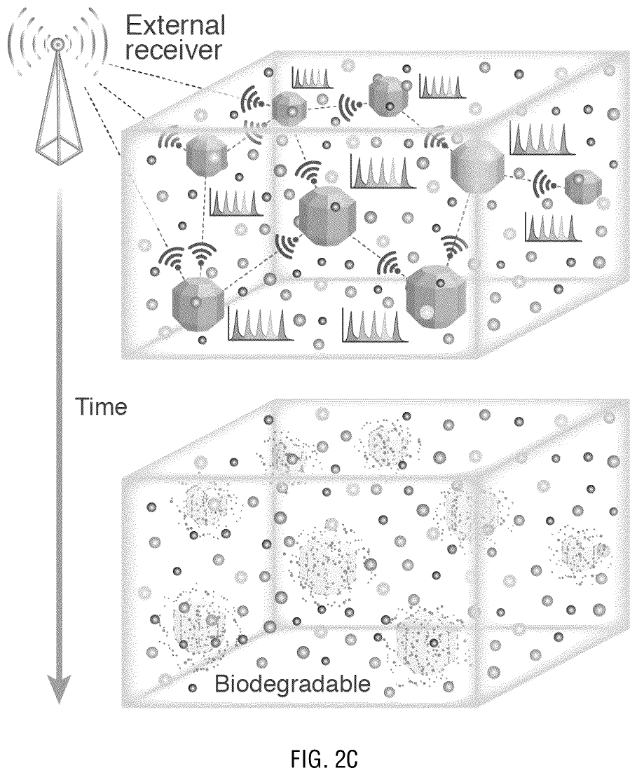

Reference is now made to FIG. 1B and FIGS. 2A-2C schematically showing the overall design concept of the present invention. FIG. 2A shows the overall architecture of the designated programmable, battery-less and biodegradable smart dust particle (SDP) of the present invention, which is also called a biodegradable mass-spectrometry-in-a-particle (SDP). FIG. 2B shows an expanded view on the main sensing layers (with uGC) and related components therein and signals obtained from each individual chemical compound received from each layer upon exposure to a complex mixture. The top image in FIG. 2C illustrates the invented SDP swarms in the environment using RF for energy-harvesting and communication between various SDPs and between the SDPs and external receiver; and the bottom image shows biodegradability of these SDPs with time at the end of their mission or activity.

The timing and condition for biodegradability are controlled by means of the precise synthesis and fabrication process of the uGC and SDPs. Thus, the current invention relates to a disruptive programmable, battery-less and biodegradable spectrometry-in-a-particle (SDP) having the size of a grain of sand that can exist effortlessly and autonomously anywhere and anytime, both on a local and global scale, with minimum human intervention (indoor, outdoor, building, human body, plants, etc.) to analyse and map in-situ the widest spectrum of both targeted and non-targeted chemical compounds (as shown in FIG. 1B) as a function of spatial coordination and time.

As stated above, the term “smart dust particle (SDP)” may be equally replaced with the term “biodegradable mass spectrometry-in-a-particle (BMSP)”, and as such, these two terms are used interchangeably in the present invention. In general, the term “smart dust particle” stands for a few millimetre-sized device that can operate as an individual component using a small or no power supply. It consists of multiple wireless microelectromechanical system (MEMS) particles ranging from micrometres to millimetres in size. “Smart dust” is a collective term for plurality of such tiny devices with extensive applications in science and technology. “Smart dust particles” are also known as motes which are equipped with sensors, cameras, and other communication mechanisms. These are ultimately connected to a computer network wirelessly to process the data procured through RFID (radio-frequency identification) technology. These minuscule devices can remain suspended in an environment similar to dust, and therefore, the collective term used herein is “smart dust”. They are suitable for collecting various data from their environment that include light, mass, vibrations, temperature, pressure, acceleration, humidity, sound, magnetism and stress. The data is transferred from one smart dust particle (SDP) to another SDP until it reaches the transmission node. The smart dust particles are designed to wirelessly process the data with an external computer system, storing the data in an external memory and wirelessly communicating the data to the cloud, a base, or other smart dust particles or MEMS devices.

The SDPs can also be considered nanites or nanobots. These terms stand for completely autonomous nanodevices or nano-systems whose components are at or near the scale of a nanometre. More specifically, nanites or nanobots refers to the nanotechnology engineering of designing and building devices ranging in size from 0.1 to 10 micrometres and constructed of nanoscale or molecular components. Such devices are largely in the research and development phase currently, but some primitive molecular machines and nanomotors have been tested. An example is a sensor having a switch approximately 1.5 nanometres across, able to count specific molecules in the chemical sample. The first useful applications of nanites may be in nanomedicine. For example, biological machines could be used to identify and destroy cancer cells. Another potential application is the detection of toxic chemicals, and the measurement of their concentrations, in the environment. Nanites are largely related to spectroscopical methods of analysis.

The SDP system is anticipated to be low-cost, scalable, and printable. Such a system would bring remarkable proliferation both as individual systems as well as network of systems that communicate between them and probably other mobile devices over large surface areas to identify health risks, optimize agricultural production, and manage supply chains. FIGS. 2A-2C illustrate the overall design principle of the current invention. The designated SDPs are biocompatible and biodegradable, and, therefore, have minimal effect on the biosphere after certain working lifetime.

The present inventors in their co-pending application have recently disclosed the nano/micro-structural arrangement made of hierarchically stacked layers of functionalized array of graphene sensors on free-standing films made of cellulose fibres (HSGC) with inkjet-printed components inspired by butterfly wing arrangement for effective semi-separation and instantaneous detection of various chemical compounds in different instants of time. This application in its entirety is incorporated herein by reference.

The concept of the current invention combines efforts to develop revolutionary sensing capabilities that improves the scientific basis for obtaining enhanced performance from sensors, spectrometers and smart dusts (SDPs), especially when targeting in-situ mapping concentration of compounds. This could give a clear improvement over the past reported implementations of spectrometry, and, therefore, allow further progress in the chosen areas of application. In addition to these, the present invention addresses important problems in chemically controlling the electrical properties of nanomaterials and devices. Indeed, the small size, biodegradability and anticipated low per-device cost of the proposed SDPs has the potential to allow an unobtrusive deployment of large and dense spectrometry in the physical environment, thus enabling detailed in-situ spatial monitoring of real-world phenomena, while only marginally disturbing the observed physical processes. The invention's approach would be used, and, therefore, impact a wide variety of application domains, including environmental protection (identification and monitoring of pollutions), habitat monitoring (observing the behaviour of animals in their natural habitats), and more.

Due to its tiny size, the SDP is expected to enable several novel applications. For example, it is anticipated that the device nodes can be moved by winds or can even remain suspended in air, thus supporting better monitoring of weather conditions, air quality, and many other phenomena. Other examples include biosensing, for example, within the human digestive tract, controlling the movements of the BMDPs in the digestive tracks using internal magnets or natural magnetosomes and external magnetic field, large-area sensing, confined space monitoring of chemical and biosynthetic reactors, oil and gas conduits, and aerospace programmes. This innovative approach for mapping chemicals in space and time would have a direct effect on existing policies and policy makers (e.g., WHO, EPA, NHS, etc.) for promoting new procedures and protocols for rapid decision at the right time before unavoidable and unwanted catastrophe happens, such as atmospheric pollution, environmental change, technological hazards, biological weapons, and ever-increasing chronic diseases.

In the first aspect of the present invention, developed are new chemically-and physically-controlled processing principles for obtaining ultra-miniaturized biodegradable particles (~500 μm in diameter) that could enable separation/elution and sensing of all individual species found in a complex mixture of volatile chemical compounds. This aspect is achieved by designing all-printed hierarchically-stacked biodegradable layers functionalized with chemically-sensitive arrays of graphene on free-standing films made of cellulose fibres (HSGC) that are equipped with all components needed for RF-wireless communication.

This aspect includes the real-time spatiotemporal separation, elusion and detection of molecules in the mixture due to different mass transport phenomena (due to their distinct molecular weight) and/or unique spin injection (due to chirality induced spin transfer) while passing through porous HSGC. This aspect is connected to the next aspect.

The results from this part of the invention (i) demonstrate the ability to tailor-make the HSGC layers and their assembly as 3D SDP design architecture, (ii) demonstrate the ability to achieve compatible and naturally degradable circuit components, and (iii) protocol for mass scale production of SDPs.

In the second aspect of the present invention, structure-property relationships of the HSGCs and SDPs and their related components are established, and their properties and behaviour upon operation are controlled. This aspect includes:
(1) Surface and structural analysis (incl. morphology and porosity) in conjugation with electrical and magnetic measurements to finely control the various ingredients that make the best sensing parameters;
(2) Layer-dependent and chemistry-dependent HSGC and SDP sensing performance to understand the detection and discrimination ability of chemical compounds in time and space, similar to the way chromatography operates;
(3) Thermodynamic analysis of the binding properties of chemical compounds and graphene's functionalizing agents and/or cellulose and modelling the mass transport kinetics within the SDP; and
(4) Density Functional Theory (DFT) and molecular dynamics simulations to investigate the influence of graphene's spin on the detection and discrimination between compounds, mainly those having similar structure (e.g., chiral molecules); and (v) bio-compatibility and biodegradability tests for in-vitro cell culture and in-vivo animal models.

The results from this part of the invention (i) enable understanding the various structure-property relationships that could explain, predict, and control the properties and behaviors of SDP systems in the environment; (ii) demonstrate all-spectrometric monitoring for wide range chemical compounds from mixtures with mm and micro-scale SDP device; and (iii) demonstrate compatible and naturally degradable circuit components to minimize the environmental pollution issue from swarms of SDP devices and that have reaching application for in-vivo implant based health care monitoring directly from inside/outside the body.

The third aspect of the present invention is an RF analogue power-harvesting and RF-based communication arrangement within individual SDPs and swarms of the like. This aspect is directed at the RF-based communication among SDP units and between SDPs and distributed and decentralized computing network. Combined with the first aspect, this part of the invention includes a thorough modelling and simulation of antenna parameters and geometry optimization with various printed biocompatible and biodegradable components within the SDP structure. It also includes exploring the optimal power induction and RF coupling to the target sensor's antenna, ways for analogue recording for resonance frequency, band width, and inductance-capacitance matching circuit.

For collective sampling of multiple distributed SDPs, swarm intelligence is adapted using various insect and animal-based algorithms from their communicating behaviours. A data fusion approach is adopted to build environment-specific empirical model with the help of machine learning, which is used for various predictive measures from possible contamination and hazards from various sources.

The results of this part of the invention demonstrate (i) printing of suitable ink formulation using 2D materials-based ink for wirelessly supplying the power to sensor and computing unit (antenna modelling); (ii) wireless transfer of collected data from all sensors mounted; (iii) microprocessor's integration and interconnection with all types of sensors would enable to share information among the dust sensor network or computing system. This part of the invention demonstrates the ability to tackle the large amount of data from billions of sensors and to reduce channel capacity problem in real time by means of distributed and decentralized computing network. Automated decision making with the aid of Al helps to make efficient data processing and early decision making about proposed strategy to combat environmental issues and humans' overall healthcare.

The fourth aspect of the present invention is a constrained environmental sensing with SDP swarms and related state-machine operation. This aspect provides a proof-of-concept demonstration on the utility of autonomous SDP swarms in mapping and tracking a whole-spectrum of compounds within a mixture, both in terms of time and space. This is done in a simulated environment in the laboratory. Hundreds or thousands of SDPs are distributed inside a specially designed chamber, into which various individual and complex mixtures of chemical compounds are spiked/distributed. The detected spectrum of chemical compounds is collected by means of RF communication from each SDP or group of SDPs, both in time and space as well as in relation to distance from the contamination source. A complementary part of this aspect includes estimation for mechanical forces on SDPs during application on surface and in constrained places and in-flight analysis while benchmarking with standard mass spectrometry.

In frame of the fourth aspect, simulated field trials of the distributed SDP devices provide constant evaluation about their efficacy, validity, interconnectivity, and spatial mapping for all (or most) chemical compounds found within complex mixture. Distributed and decentralized computing network provide gigantic dataset in the global scale and real time. The system continuously tacks the detected chemical compounds in each spatial spot, both in-situ and in real-time. The nature of the detected chemical compounds is identical to those of conventional GC, thought SDP exhibits superior performances in terms of tracking and chemical mapping capabilities. For all of these, the SDP swarm would help to make efficient data processing and earliest decision making about proposed strategy to combat environmental issues and humans' overall healthcare.

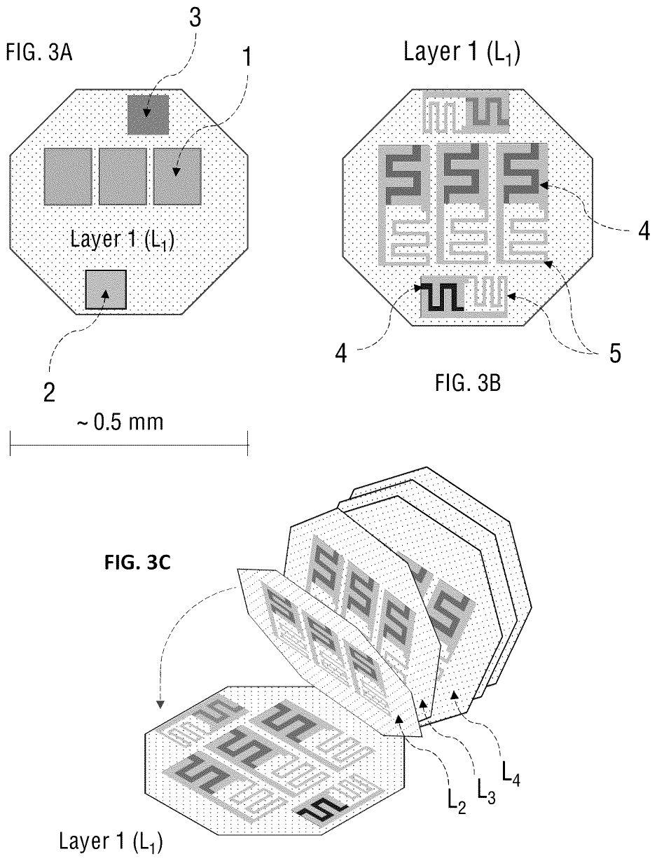

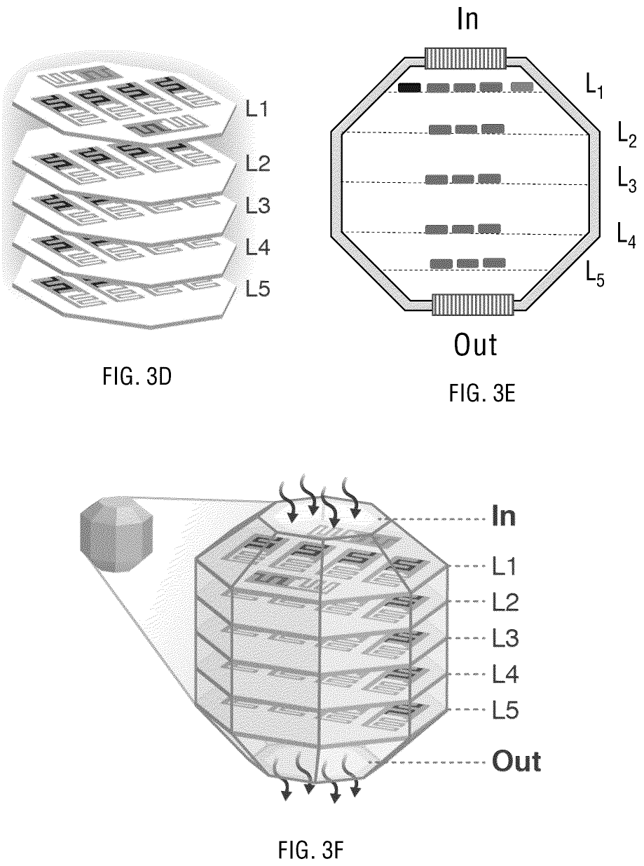

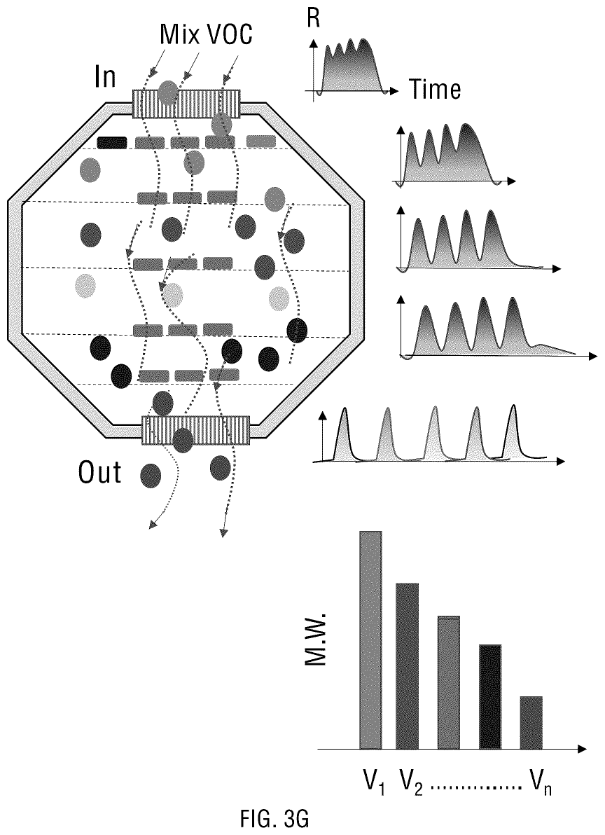

One embodiment of the present invention relates to technical implementation of the first aspect mentioned above. According to this embodiment, the design and operation of HSGCs and SDPs together with pertinent biocompatible electronic components that could degrade at a natural or physiological environment by the end of the task, in a way they do not pose an environmental hazard are shown in FIGS. 3A-3G. The fabrication of the SDP with the incorporated HSGC is described by the following steps.

### Step 1—Synthesis and Printing of Biocompatible Sensors

Mass scale printing of biocompatible ink on biocompatible and porous substrates, such as, cellulose fiber-made paper, is carried out using piezoelectric inkjet printer (pico-liter resolution) with binder-free 2D nanomaterial-based inks (such as, graphene) or metal nanoparticles (gold NPs), without the need for post-processing annealing and/or stabilizing agents or process. The ink formulation is based on bioinspired adhesive protein (polydopamine) like mussels' protein that inherits natural adhesive property for wide range substrate even in underwater. This serves as simultaneous reducing agent, binder, and surfactant of 2D material-based ink with variety of grafted biochemical ligand for various additional functionalities. Various thiol/amine-terminated biochemical ligands with variety of functional groups, such as, —COOH, —OH, —Cl, —CH₃ and chiral center were prepared and examined.

The selection of specific ligand is finalized based on cytotoxic assessment of various cell cultures and bio compatibility experiment (cf. above second aspect of the present invention). Printed sensors using gold nanoparticles (3-5 nm in diameter) that are modified with various thiol-based moieties, after relevant biocompatible assessment, are explored as well. Temperature and humidity sensitive sensing layers, using carbon-nanotube ink solution and natural silk protein solution, are prepared and included in the HSGC layers.

Reference is made to FIG. 3A schematically showing the printed layout of an SDP of the present invention that comprises sensors (1) for gas compounds (these sensors are made from biochemically modified graphene/gold nano particles), temperature sensor (2) (made from CNT ink) and humidity sensor (3) (made from silk protein ink).

### Step 2—Printing Electrodes and Antenna

Interdigitated electrodes and antennas are printed by biocompatible conductive inks that contain silver nanowires, magnesium or zinc nanoparticles, using similar approach to that mentioned in Step 1. FIG. 3B illustrates the printed electrodes (4) and antennas (5). The selection of the best printed electrodes is made by comparing their electrical performance to similar ones made by thermal or e-beam evaporation.

Since these components act as single impedimetric device that are powered and communicated by specific external RF source, in-depth simulations are carried out, mainly for the antenna part, to examine the RF-related parameters (dimensions, resistance, capacitance, band width, resonance shifting, multi band operation, power optimization and suitable coil design) for optimizing the energy-harvesting and experimental iteration to communicate the device remotely. This information is iteratively optimized by modulating the fabrication and synthesis condition of sensing, electrode and antenna biocompatible materials.

### Step 3—Mass Scale Fabrication and Layered Integration

All printed components mentioned in Step 1 and 2 are applied for mass scale in larger sheet of cellulose paper, as seen in FIG. 3E schematically shows the cross-section of the SDP with a protection layer. An example is shown in FIG. 3C and FIG. 3D illustrating the multitask arrangement for each printed layer in hierarchical arrangement. The fabrication is carried out by stacking all printed HSGC layers in multi-stack arrangement (see FIG. 3D for a single device and FIG. 3E for mass-scale printed paper).

Each printed sensor is hierarchically arranged in layer-by-layer, from bottom to top, as shown in FIG. 3F illustrating the 3D-printed protection layer encasing the SDP unit. The circumference of each hexagonal HSGC layer is sealed with proper adhesive. Finally, the top layer is equipped with additional porous layer shown in FIG. 3F encasing the SDP unit with proper sealing in the periphery. FIG. 3G shows the separation of the VOC components V₁, V₂ . . . Vₙ in the mixture based on their molecular weight (M. W.) with the SDP of the present invention.

### Step 4—3D Nano Printing of Biocompatible Protection Layer

Due to the anticipated mass scale injection of millions of nanites (SDPs) in the environment, the toxicity and privacy-related effects from the SDPs and their components may be a concern. Therefore, biocompatible electronic components that has the ability to degrade and dissolve naturally at a normal or physiological environment by the end of the task are incorporated in this step. Towards this end, an additional protection layer made of various biocompatible materials, such as, polylactic acid (PLA), poly(lactic-co-glycolic acid) (PLGA) and polycaprolactone (PCL), mostly in concentration of 50 mg/mL, are used as to protect all over the device generated in Step 2-3 above by keeping an open channel in top and bottom for passage of chemical compounds.

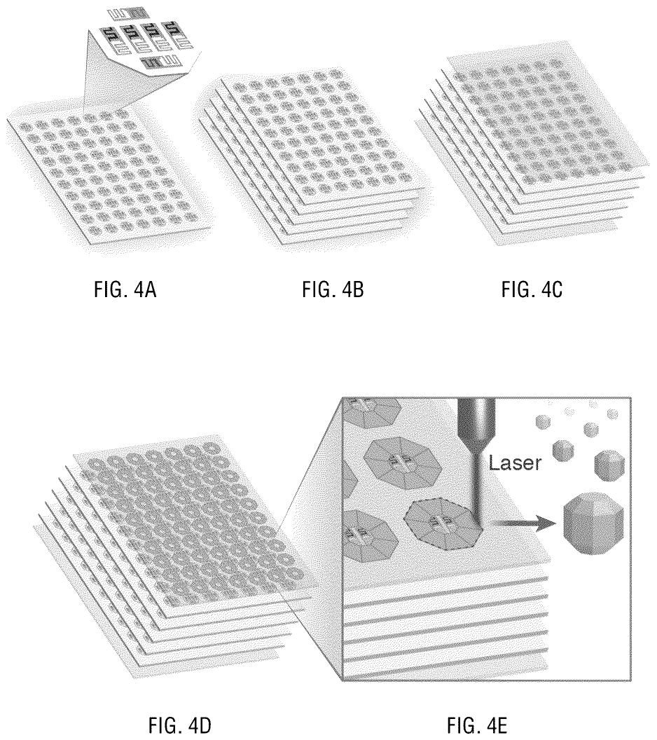

Reference is made to FIG. 4A schematically showing the design of the mass scale printed sensor and antenna electrode on a sheet paper, FIG. 4B illustrates the layered arrangement of each layer, and FIG. 4C shows this design with the top and bottom porous layers covering the separation layers. FIG. 4D demonstrates the 3D printing of the protection layers on a mass scale. 3D nano printer is used to create the protection layer and to integrate all types of sensing components in layer-by-layer hierarchical architecture.

Finally, a laser-based cutting instrument is used for dicing individual dust device as shown in FIG. 4E illustrating the dicing of an individual SDP of the invention. This transient electronics-based biodegradable SDPs have the potential to be used for both environmental sampling as well as implant-based monitoring for human and animals. Yet, in extreme conditions (e.g., weathers) and mechanical friction, these devices have to compensate for accidental rupture in the SDP's wall during the whole time of the targeted task. To solve this issue, all micro-circuit components are mounted and packaged via self-healable biocompatible material, made of prepolymer-cetyltrimethylammonium bromide (PUIDE-CTAB) elastomer, following the design and protocol which is intentionally undisclosed here.

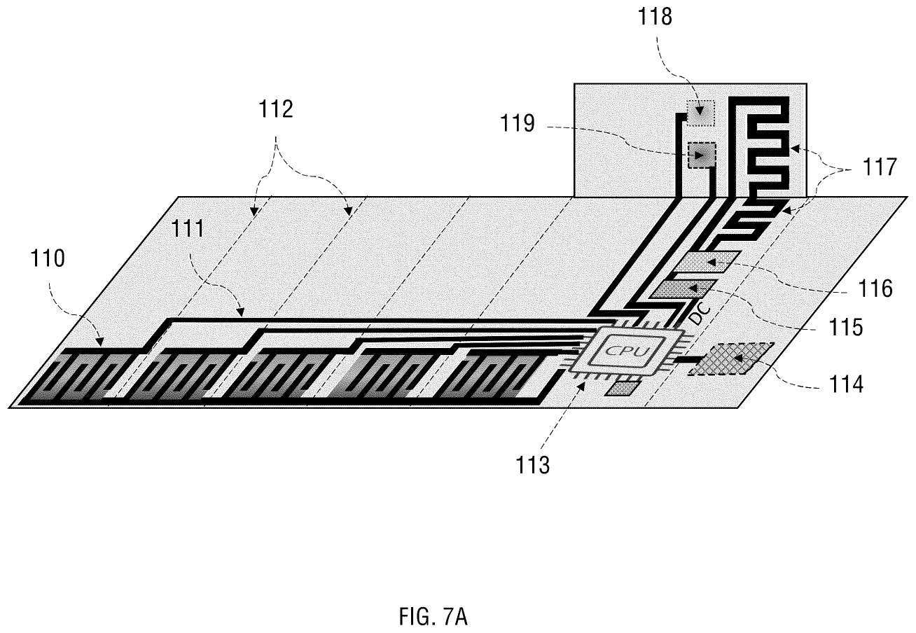

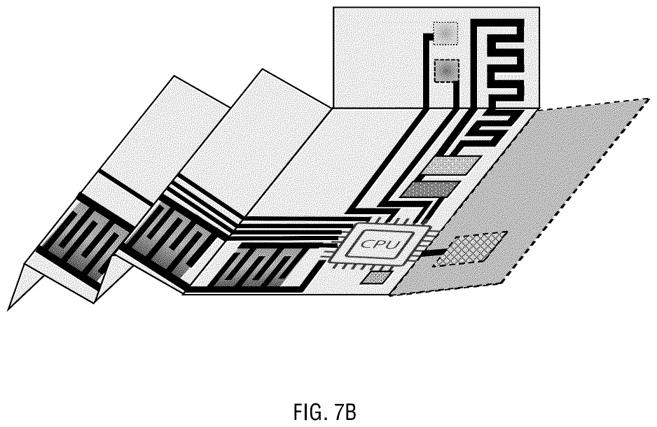

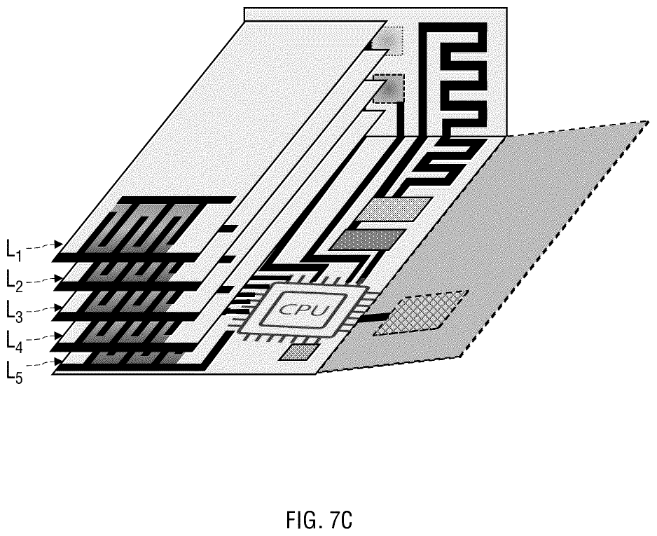

Reference is now made to FIGS. 7A-7E schematically showing the entire printed system of the present invention comprising the RF powered mass spectrometry on the all-printed smart dust sensor in the folded layer-by-layer design, and it is sealed. FIG. 7A shows the system in the unfolded state, where 110 is an inkjet-printed sensor, 111 is an inkjet-printed electrode, 112 are folding lines, 113 is a microprocessor, 114 is a transmitting antenna, 115 is a printed filter, 116 is a printed rectifier, 117 is a printed radio-frequency (RF) receiving antenna, 118 is a temperature sensor, and 119 is a humidity sensor. FIG. 7B shows the system being origami-folded along the folding lines 112, and FIG. 7C shows the semi-folded system after folding the layers. FIG. 7D shows the semi-folded system with the top porous layer 120, and FIG. 7E shows the completely folded and sealed system of the present invention.

An alternative approach to combine these capabilities would rely on standard Micro-Electro-Mechanical Systems (MEMS) fabrication techniques, together with two-photon polymerization (2PP) 3D printer technology.

### Step 5—Evaluation of the SDP Performance

In this part of the invention, a time-space resolved geometry is developed by utilizing a micro-origami like folded porous architecture, where the sensors in each layer is facing a time lagged sensing profile like a conventional chromatography column (GC). This layered architecture generates a GC-like separated sensing profile due to unique transfer rate of each compound in each layer with better accuracy and reliability. As such, the SDP and related HSGCs are benchmarked with standard GC using laboratory based simulated mixed gas environment.

This experiment was performed in variety of temperature (−20 to 60° C.) and humidity condition (~0-100% RH) to mimic real situation in a simulation chamber. All printing materials used in this study are preselected and optimized by their respective structural, spectroscopic, rheological, electronic, microscopic, and biocompatible characterisation. With on-board printed temperature and humidity sensor, best temperature/humidity calibration sensitivity and stability for long run was tested and optimized. The SDP was predominantly developed by identifying various specific chemical compounds, such as methyl bromide, 1,3-dichloropropene, metam-sodium, chloropicrin, metam-potassium, glyphosate, etc. The detection of such complex compounds was simultaneously monitored by in-built SDP and various other biochemical ligands grafted on the HDGC sensors. The specific chemistry on the surface is highly beneficial for recognizing and detecting such complex compounds. This step is carried out in tight conjugation with in-depth structure-property schematizations mentioned in the second aspect of the present invention.

### Step 6—Quality Control and Minimization of SDP Variation

The fabricated devices in large scale need to perform similar in same experimental configuration. Therefore, statistical analyses are carried out to relate device fabrication defect or anomaly with device fabrication parameters and obtained results from similar sensing event. The obtained model is used and verified to test mass scale device to preselect rapidly the best device for final selection. Although visual inspection of each individual device is very sluggish to quality control, RF performance and relevant sensing profiles is determined to accelerate the preselection of best SDPs. This will help commercialization or industrialization of these devices for continuous production and rapid quality control.

### Surface and Structural Analysis

Various surface and structural analyses were carried out for the SDP and its related components: Atomic Force Microscopy (AFM) to determine the thickness and surface topography of the HSGC layers; Scanning Electron Microscopy (SEM) and Cryogenic Transmission Electron Microscopy (CryoTEM) to examine the morphological features; Fourier Transform Infrared (FTIR) and Raman Spectroscopy to determine the presence of biochemical ligands and structural information in the HSGC layers; Brunauer-Emmett-Teller (BET) surface analysis to determine the adsorption/desorption isotherm as well as the porosity characteristics of the HSGC layers.

The output from these characterizations correlates with the fabrication process described above as well as with the additional characterization techniques mentioned below, to achieve the highest possible SDP performance.

### Electrical and Magnetic Measurements

This included electrical measurements, such as resistance, capacitance and impedance parameters, using Graphical SourceMeter (SMU) and impedance measurement system for various frequency ranges. These parameters directly correlate with the process parameters (synthesis and fabrication) and are tuned as per design parameters for the final SDP. Magnetic influence of the spin injection effect during exposure to various chiral and non-chiral volatile compounds (similar to those mentioned below) to explore the inter-relation and influence of various biochemical ligand (with/without chiral center) was carried out by Magneto-Raman spectroscopy, in-situ magneto-resistive measurements and quantum Hall measurements.

### SDP Sensing Performance Upon Exposure to Individual and Mixtures of Chemical Compounds

Each of the HSGC layers in the SDP device were characterized according to its position in the hierarchical HSGC stack upon Direct Exposure (DE) to various chemical structural/chiral mixtures, with and without the spatiotemporal part. Examples of structural/chiral mixtures that used in this characterization include methanol (0.1 ppb to 500 ppm) and pure/mixed vapors of ethanol and isopropanol (mix ratio of M:E:I~0:0:1, 0:1:0, 1:0:0, 1:1:1, 2:1:1, 1:2:1, 1:1:2). For each layer, the sensing results, the response kinetics, and effect of time-space resolved architecture of HSGC was explored. Based on the outcome of the results, the HSGC architecture was challenged with additional complex mixtures of 10-100 analyte types (alcohol, aldehyde, ketones, hydrocarbons, organic acid).

The time-space resolved complex data set was used further as sequence input to machine learning-based Neural Network Architecture (DLN) for generating and predicting a performance that resembles the compounds elution from areal chromatogram. For this purpose, a typical sequence input layer to DLN, fed from the HSGC derived time space resolved data, was used. This specific time-resistance sequence was further processed to Short-Term Memory (LSTM) layer and fully connected layers.

Finally, a regression layer was added in the end to predict the entire chromatogram-like performance for continuous prediction of the chemical compounds. Due to the large data set, a wavelet-based signal processing was used to compress the time sequence data and then used for deep neural network's sequence input layer to minimize the running time and memory size. For mixture states, image processing was used by synthetically constructed input image from HSGC data and fed to the self-learning architecture of deep net layers.

Deep net system automatically samples the features from same size image by itself and used for learning to classify the various mixed chemical compounds. The calculated result was used for the hidden layers to show the best prediction for other mixtures. Complementary to the spatiotemporal part, loading the hybrid FDrGO sensors was examined with various biochemical ligands that exhibit remarkable sensing difference between each single chemical compound and their mixtures.

Amongst the chemical mixtures that were examined in this process, the detection, separation, and discrimination between several mixtures of enantiomers and chiral molecules were challenged. So far, using the current available techniques, these molecules were rather difficult mission to achieve. This could be due to insufficient chiral atmosphere at the host device, which, sometimes, is not enough to resolve a racemic/enantiomeric mixture easily.

As a demonstration example, the response pattern for pure (0:1, 1:0), racemic (1:1) and enantiomeric (2:1, 1:2, 3:1, 1:3) mixture of S (+) butanol and R (−) butanol was examined for various achiral and chiral ligands (e.g., mercapto-hexanol, diethanolamine) respectively. The originating features, in terms of orientation of resistance change modes, such as, up-down, down-down, down-up and up-up configuration were examined and analyzed as a way for robust discrimination of enantiomers.

### Binding Analysis of Gas-Biochemical Ligand at Sensor Surface and Gas/Cellulose (from HSGC)

Binding strength and potential splitting phenomena of gas-ligand and gas-cellulose was examined for representative chemical compounds, using molecular docking and DFT. Of special interest was the challenging racemic/enantiomeric molecules and their mixtures, such as cellulose-S(+) butanol and cellulose-R (−) butanol, while passing from one layer to next. The interaction energy and various thermodynamic parameters of gas-solid complex was calculated to explain the relative interaction strength, which is related to sensitivity of the device, and spatiotemporal splitting of mixtures, which is related to the helical cellulose interaction from paper that acts as a stationary phase similar to a chromatography.

To understand potentially opposite orientational changes in chiral vapor exposure, ab-initio DFT analyses for optimized structure was used to calculate HOMO-LUMO gap and thermodynamic parameters for each case. Although these calculations could explain the interaction strength of each complex type, the spin polarization effect originating from the molecule-induced spin selectivity effect (CISS) was further studied by means of charge injection accompanied with specific spin orientation from specific gas molecules. The theoretical study was supported by magneto-resistive measurements in constant flow of chiral molecules. NMR data was used for comparison of specific ligand center for a biochemical ligand on graphene surface.

### Theoretical Modeling to Investigate the Mass Transport Through the HSGC Layers

To fully understand the multi-component separation and detection by SDP, a theoretical kinetic model was developed to describe the gas separation and sensing event using Langmuir adsorption-desorption kinetics. Since each component of the gases enters, reacts, and leaves the specific layers, a superposition model for multi-gas kinetic model was used to describe the presence of local maxima of the non-linear resistance profiles with respective fitted area using Gaussian/Lorentzian multipeak fitting modelling.

The entire resistive chromatogram-like structure was then compared with various simulation or real sample analyses using gas chromatography spectrum analyses. Kinetic modelling provides a unique generalized time dependent equation that describes the injection of specific gaseous chemical compound to a layer, reaction to surface and release to next layers in superposition model of multi-components where each peak uniquely identifies as retention time of certain chemical compounds in similar manner to standard gas chromatography. This equation was further reformed to include the various effects, such as porosity, flow rate, viscosity, and inter layer distances, to represent a complete unification of overall effect and various parameters in the structure of the SDP format.

### Dissolution Tests for SDP

The test structures for studying dissolution behaviours of SDP devices were caried out by immersing into ~20-50 mL of phosphate buffer solutions (PBS, 0.1 M, pH 4-10) at a temperature of −20 to 60° C. The samples were removed from the solution at regular time intervals to measure the their structural, electrical and sensing properties till final disappearance/biodegradation of the SDP devices.

### Biocompatibility Tests in In-Vitro Cell Culture and In-Vivo Animal Model

The cell cytotoxicity of SDPs was examined in various healthy cell lines, such as human heart cell line (AC16 Human Cardiomyocyte Cell Line), liver (Human Hepatocytes cell lines), skin (epidermal skin cell lines), kidney (parietal epithelial cells) for various dosage amount (concentration in the assay) and treatment time. In parallel, cytotoxicity experiments were carried out on individual circuit components to establish the full biocompatibility investigation and scrutinize the optimal materials selection. Ingestion-based study on animal model by intaking these devices through food was carried out in rats, like the protocols reported elsewhere. Epidermal skin biocompatibility test was assessed for allergic or other types of health disorder on shaved skin of rat.

### RF Analog Power Harvesting and RF Based Communication Arrangement in Swarm Mode

An inclusion of a battery for powering the SDP would largely determine the overall size of the SDP and bring it to dimensions that deviate it from the anticipated vision. In addition, the constant replacement of the batteries is undesired and causes additional economic burdens and environmental problems, due to the toxic nature of battery chemicals. Thus, a battery-less power harvesting unit is required for lightweight, independent, and maintenance-free operation. Wireless RF technology can stand out as an attractive alternative to batteries while providing simultaneously communication and data transmission capabilities. RF powered scheme is a game changer as it does not need any continuous mechanical/chemical input and could be readily used from a RF power station wirelessly.

In a certain embodiment of the present invention, an RF power-receiving system, receiving antenna coupled with impedance-matching printed circuit, printed diode-based rectifier, and capacitive filter for converting incoming AC RF signal to usable DC power to propel the device are designed.

An RF signal generator was programmed as per matching transmitted signal value and an antenna modelling was carried out for correlating and optimizing the various antenna parameters, such as, length, conductivity, gain, power conversion, resonance Q-factor, ripple factors, etc. This way, the RF-based SDP could collect the energy from an external remote power generating module without need of in-chip battery or other self-power-based schemes, such as a triboelectric generator where a continuous mechanical agitation is needed to continuously power the device. In the course of all of these studies, a special research attention was focused on using the conventional mobile/cellular towers' power distribution to the millions of SDP devices distributed everywhere.

### Antenna Fabrication and State Machine Operation for Power Balance

The SDP devices were self-powered by in-built antenna. The antenna was optimized for a non-stop data acquisition mode when RF pulse hit the SDP device exclusively. A rigorous modelling with suitable optimization of the antenna fabrication (dimensions (20×10×1 mm); conductivity; inductive couplings; resonance shifting; bandwidth modelling; resistance-capacitance matching; impedance modelling; etc.) was carried out to project the micro-state machines as a true remote accessing tool. This led to an estimation of the power harvesting in the circuit as well as its dynamic recording from modulating the overall impedance network (sensor+antenna) with a standard receiver. This overall remote access was done in various dynamic simulated environment for in-flight and precipitation mode that undergo most relevant flow regimes, i.e. from laminar, via transitional, to fully turbulent. In addition, the distance between SDP and the RF source was optimized for maintaining the best power balance and analogue data access.

### RF-Based Data Acquisition on a Large Scale

RF-based wireless data transfer strategy was implemented through an inductively powered micro-passive SDP that was directly used as powering communicating device. A master processor-based device that collectively and remotely communicate with multiple SDP devices was used. All processed data, from all SDPs, was directly accessed and transmitted to the nearest station and GPS. This could be achieved by a moving drone or via nearby station to scan a wide area. For many applications, a very large number of distributed sensors and related feedback from master processors, simultaneous data processing and transmission face problems due to limited channel capacity. To solve this issue, swarm intelligence-based routing algorithm was implemented for large data processing and reducing channel capacity problem from millions of implanted sensors through a wireless sensor network (WSN).

Predominantly inspired by biological systems, swarm intelligence are usually a projection of insect and animal-based algorithms from their communicating behavior in minute detail. This could be ant-colony optimization algorithm or bee-based communication, firefly networks algorithm or wolf based inter-communication and analyses. Such distributed and collective behaviour from any specific place of interest would be used further by machine learning protocol for automated learning in detail. The acquired model from such ground level data from both human and environment was used in the future for faster decision making that was highly useful for preventive measures before a large-scale catastrophe.

This distributed and decentralized computing in conjunction with machine learning would be highly beneficial for understanding and modelling the gigantic data set more easily than currently possible. It will further confront the challenges in the SDP-based WSN field, such as, large-scale networking, dynamic nature, resource constraints, and the need for infrastructure-less and autonomous operation that will operate on the capabilities of self-organization and survivability.

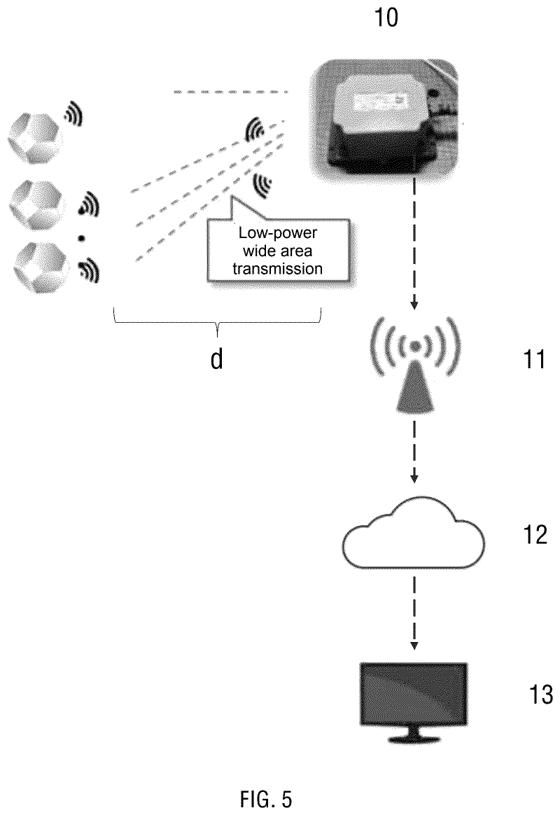

Reference is now made to FIG. 5 schematically showing the communication architecture with one shown cluster or with multiple clusters, where (10) is a mini-gateway, (11) is an access point (Wi-Fi or cellular), (12) is Internet (server or cloud), (13) is apps and data visualisation, and d is a distance of hundreds of metres up to several kilometres (depends on the communication technology used, for instance GSM, GPS or similar).

In a certain embodiment of the present invention, the sensing module is enhanced with a communication module which has the capability of transmitting in a wide range (from hundreds of meters up to some km, depending on the line of sight, transmission power level, protocol and propagation conditions). Transmission can use low-power wide area protocols which have the advantage of trade off data rate for sensitivity with a fixed channel bandwidth, e.g., 125 KHz of bandwidth. Moreover, power lifetime of the communication module can be expanded to more than a year, but the exact lifetime depends on the amount of data transmitted, on the distance, on the transmission intervals and on the technology/protocol used for transmission.

Also, there is need for a device (mini gateway) that will collect the measurements of multiple SDPs and transmit them to an access point either Wi-Fi or cellular (depending on the area's availability and the technology that we would like to use). Mini gateway acts as an aggregator and will receive data from multiple sources which can be transmitted then to the cloud. Through this approach it is possible to collect remotely the sensed data and feed useful applications and/or proceed to data analysis and visualization in various devices.

In case multiple clusters of smart dusts are utilized, the following approach can be considered. Each cluster of smart dust particles can transmit to a dedicated mini gateway which in turn transmits to an access point (same or different for each cluster) in order to send the data to the cloud.

In case multiple clusters of smart dusts are utilized, the following approach can be considered. Each cluster of smart dust particles can transmit to a dedicated mini gateway which in turn transmits to an access point (same or different for each cluster) in order to send the data to the cloud.

### Process and Algorithmic Optimization for Multi-SDP Access by Minimizing Reader Collision

Due to the large number of scattered devices in a common interrogation area, a reader collision is an issue due to overlap and crosstalk. To solve this issue, various anti-collision protocol as per ISO 15639 standard that use certain multi-access methods for identification to physically separate the transmitters' signals are adopted. This includes Space Division Multiple Access (SDMA), Frequency Division Multiple Access (FDMA), Code Division Multiple Access (CDMA) and Time Division Multiple Access (TDMA). Out of all protocols FDMA could be highly useful as it can selectively access a specific dust which is tuned to certain frequency.

Thus, for each SDP device, data could be retrieved using multi-band frequency transceiver and multiplexer. Using a drone or a static substation these data could be retrieved for a specific scan interrogation area and by continuously changing the location of interrogation a wide range area could be scanned.

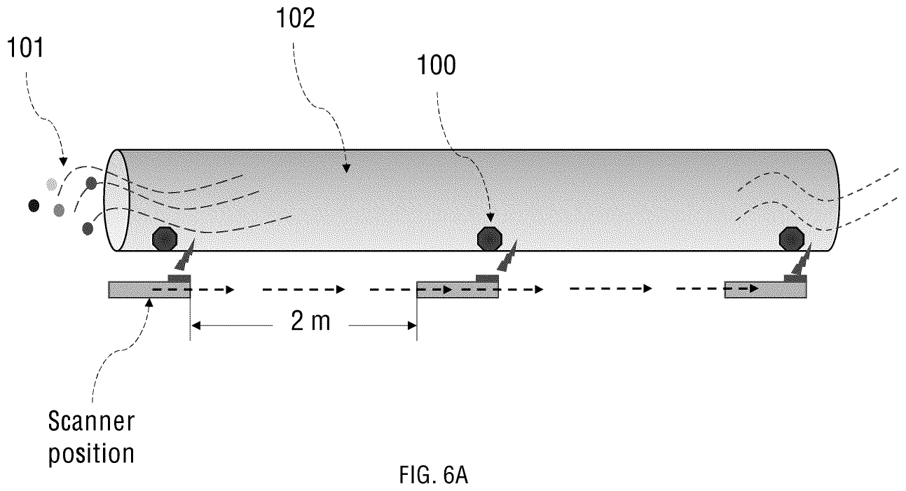

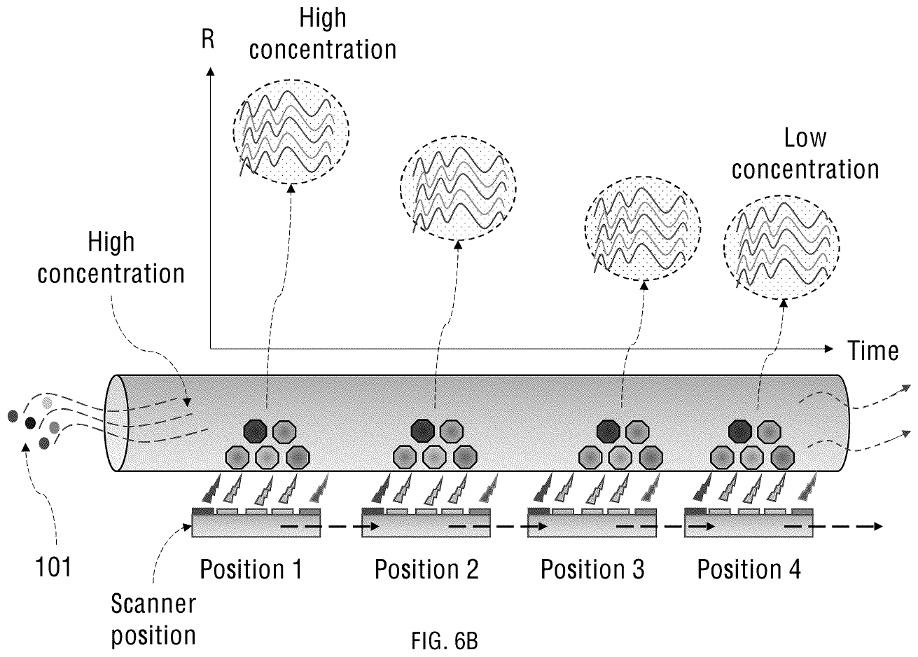

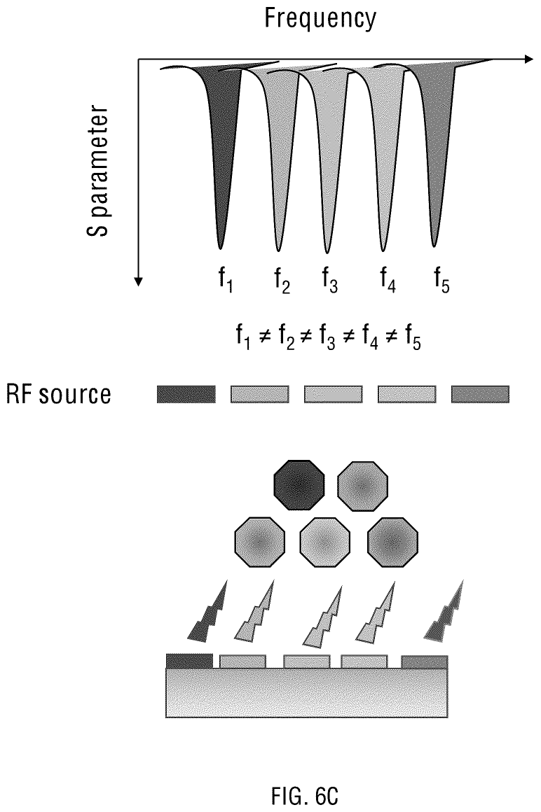

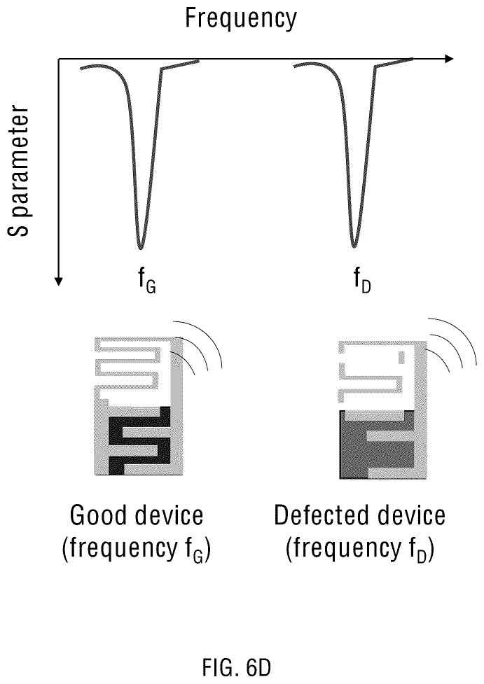

The concept is schematically shown in FIG. 6A schematically shows the experimental arrangement for the SDP devices (100) for the gas (VOC) mixture (101) sensing inside the simulation chamber (102) using multiband and single frequency measurements scheme when the single SDP units are kept at a longer distance. FIG. 6B schematically shows the experimental arrangement for the SDP devices for the gas (VOC) mixture (101) sensing inside the simulation chamber using multiband and single frequency measurements scheme when the multiple SDP units are placed very close to each other, and the concentration mapping during the elution of the mixture from the simulation chamber.

FIG. 6C schematically shows the principle of the operation of the SDP devices, where an RF source represents a spherical waveform reaching all the SDPs simultaneously. Consequently, a frequency tuning as shown in FIG. 6D must be done to take the data wirelessly. Thus, using each time sequence, the collected data is processed to swarm protocol to check the interdependence between each time-space dust cluster under scanned interrogation area. In case of detecting certain threat, the collective response is monitored, and a rapid action will be taken to resolve the issues. The procedure could be used for healthcare monitoring as well.

### Wireless Information Recovery, Consolidation, and Contextualization From a Swarm of SDPs

In this aspect of the invention, the RF-based communication and remote powering of a swarm of SDPs was studied and developed, allowing for the capture, recovery, and annotation of data. This part of the invention addresses the challenge of ultra-miniature antennas of the SDP, both in passive (resonators coupled with the antenna) and active (sensors accessed via electronic IC) modes. This also addresses the challenge of providing enough energy to a multitude of SDPs forming a swarm, while collecting the acquired data from the individual SDPs or swarm segments of variable size.

The performance limits for RF antennas were established in confined volumes, which are less than 0.5 mm. Maximum number of sensors was defined that can be integrated into an SDP (20 sensor in the passive mode; and 50 sensors in the active mode). The maximum range of the SDP-Reader's link (1 to 10 m) was defined, and the wireless communication from more than 105 sensing nodes to multiple readers was established.

### Remote Powering With Miniaturized Receiver Antenna

As mentioned above, inclusion of a battery for powering the SDP would affect the overall size and would cause several economic and environmental problems due to the toxic nature of battery chemicals. Thus, the present invention is directed at design, and development of the SDP sensing nodes that use the energy provided by incoming electromagnetic waves to ensure the read-out of the sensors and the transmission of the resulting data to the reader node. The main challenge resides in the size of the antenna, which influences both the coupling and the selection of carrier frequencies.

The coupling is decreasing with the reduction of antenna size, while the optimal carrier frequency increases. Only high carrier frequencies will permit the miniaturization of the antenna, but the propagation losses also increase with the frequency which will require both careful selection and EM modelling. When considering an implementation, the harvested energy will require transformation to power the analogue read-out of sensors. This is typically handled by a PMU (Power Management Unit) but the extremely low received energy will challenge the use of existing solutions. A tightly combined implementation of the harvester, PMU and analogue front-end is therefore explored in the present invention in order to reach performances beyond state of the art.

### Communication Between the Sensor and the Reader

Passive SDP implementation: This part of the invention explores the possible architectures to implement a passive read-out of the sensors. Spectral encoding of the information is the route of choice since a passive structure cannot handle any kind of communication protocol. The necessary information (SDP ID and sensors readings) is encoded using a single radiative structure with multiple resonators each of which can be dedicated either to a bit encoding or to a sensor readout.

Active SDP implementation: A parallel route is carried out to power and communicate with the SDP using a semiconductor device. The integration of a miniaturized die (25 μm thick, inert and non-toxic) on the substrate and the management of the energy will be the main challenges. Importantly, this approach also enables operation of the sensor elements at optimum operating frequencies (DC to kHz). The antenna and RF propagation channel are viewed jointly and closely co-designed with the materials.

The number of sensor nodes accessed in the present invention (millions) is orders of magnitude greater than the state of the art (thousands) currently found in UHF RFID, in which the reader can see multiple tags almost simultaneously. Spatial, time and frequency multiplexing are considered together with data encoding and modulation schemes to push beyond the limits of the state-of-the-art. Depending on the architectural implementation (passive or active) of the sensing node (SDP), the communication scheme differs. When the complexity (intelligence) of the sensing node will be limited like in a passive implementation, the possibility to use specific distributions of sensing nodes configurations (by construction) is explored to reduce overlaps in time or frequencies. This is implemented in the design of complementary separation algorithms pushing back the complexity at the reader level.

The range is the second biggest challenge of the communication link. Due to the extreme miniaturization of the SDP antenna, the returned signal will considerably diminish, requiring exploring new approaches like combining the contribution of multiple nodes. Using a communication scheme allowing an intrinsic averaging of the sensed data together with an increase of the back scattered energy is explored in the present invention. For example, the spectral encoding of the sensed values (one band per sensor) is a promising approach despite the sensitivity to environmental conditions (absorption/reflections).

### Agile Beaming Capabilities

Accessing a swarm of sensors (thousands) with a single medium (communication link) poses enormous challenges in terms of messages collisions and ranges limitations. Since the sensing node is of limited complexity, it cannot handle a complex protocol of communication. To address this challenge, the number of sensors addressed at the same time by scanning the swarm of sensors using a highly focused beam is restricted.

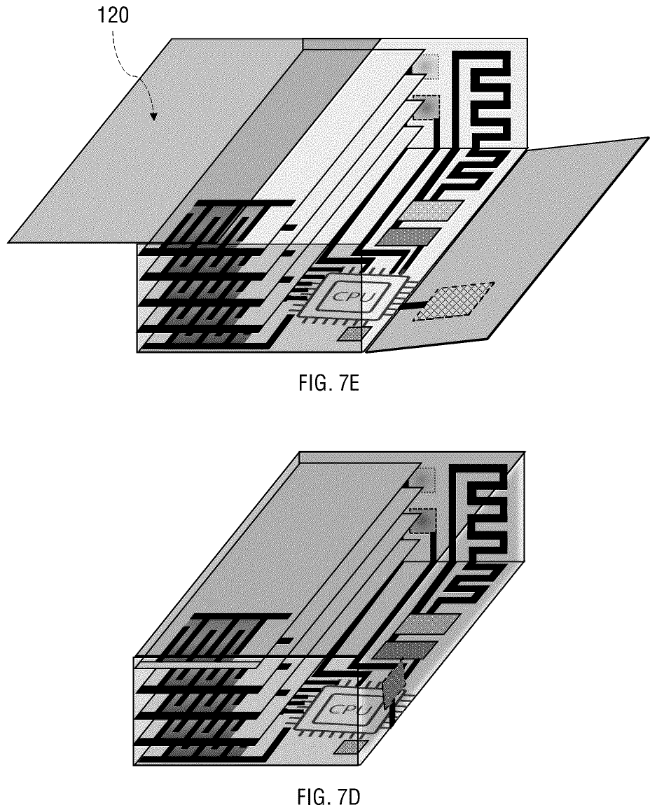

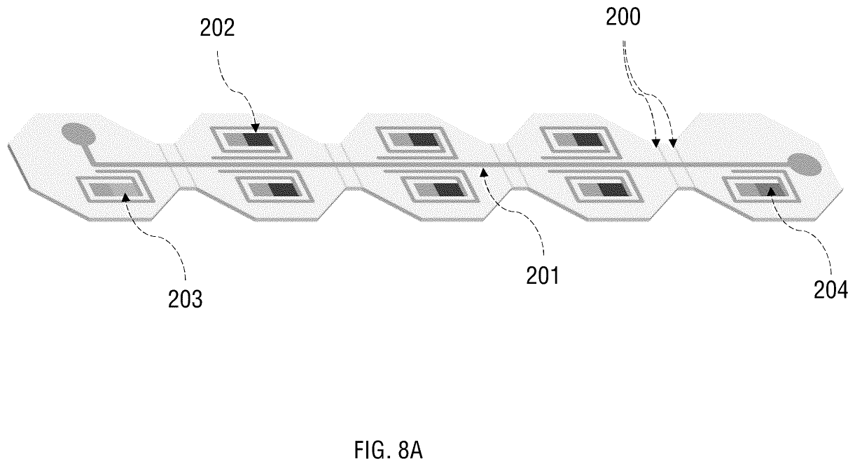

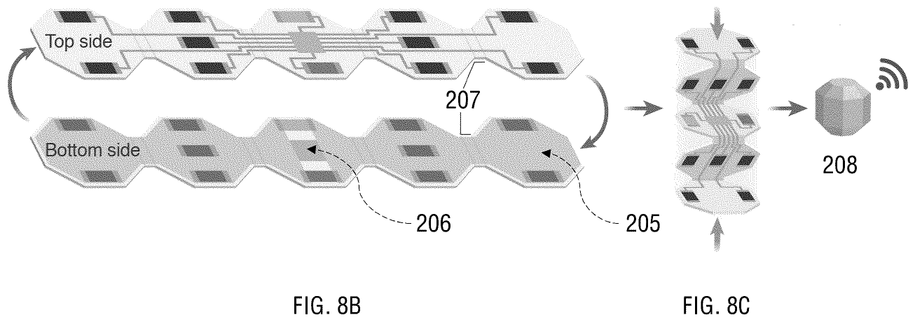

Reference is now made to FIG. 8A describing the SDP structure of backscattering antenna in a passive implementation with sensors tuned resonators, where 200 are the folding lines, 201 is a connecting wire to sensors, 202 is a VOC sensor, 203 is a temperature sensor, and 204 is a humidity sensor. FIG. 8B illustrates the SDP structure in an active implementation with the Si-based energy harvesting, sensor redout and communication, where 205 is a sensors' ground and antenna, 206 is a silicon DIE (25 μm), which decouples the sensing from the communication, and 207 is a folding gap, and FIG. 8C shows the stacking of the layers, followed by creating the smart dust particle 208 (SDP or SDP) of the present invention.

The scanning is performed by beamforming solution (antenna and baseband processing) for a static reader, but can also be implemented using a narrow beam antenna mounted on a mobile reader (drone). Beyond the improvement of the communication scheme, this approach also focalizes the energy transmitted to the nodes, helping to reach the critical level needed to perform measurements and return the resulting data. A beamforming solution is more likely to fit in static readers because of the size and necessary infrastructure. Information fed back from static and mobile readers will require strong algorithms to convert the varied spatial clues into a coherent spatial representation of data sources. Beam forming can be achieved using phased array antennas where the phase of each antenna element is controlled with a dedicated phase shifter-controlled electronically. The resulting direction and aperture can be steered very rapidly allowing to scan the area in which a swarm of sensing node has been deployed.

### Long-Range Connectivity

The readers support their own communication network (between them) and the reporting to the cloud. To achieve this, a secondary communication link providing long-range and variable data rates is integrated into the node. A specific multi-interface control protocol is designed and prototyped to handle both the locally dense data exchanges (localization, consolidation, and pre-processing) and the long-range but low data-rate reporting to the cloud. It comes on top of the communication interfaces installed on the reader (cellular, Wi-Fi, V2V).

### Network Clustering and Connection to the Cloud

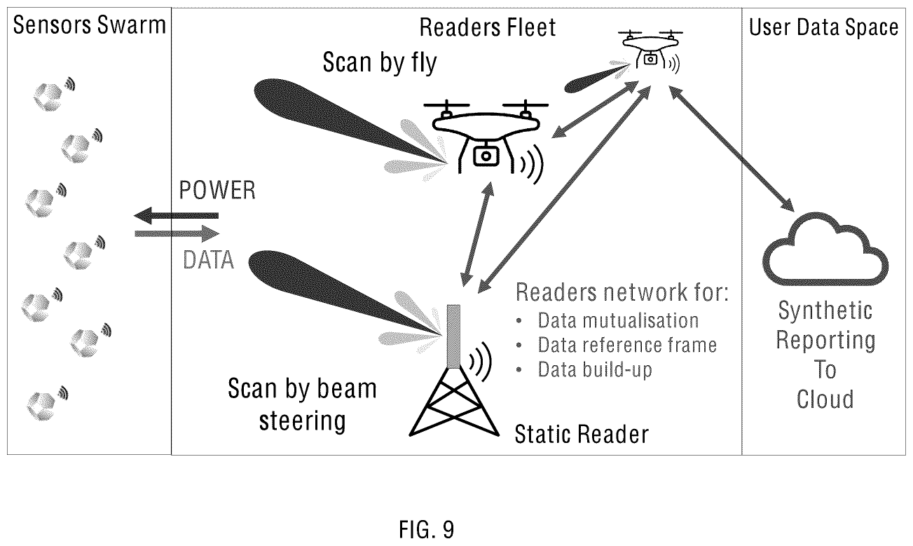

The connection to the cloud should be as direct and as simple as possible. Ideally, there is only one physical device that interconnects the swarm of sensors with the cloud, i.e., the reader would have a cellular interface. FIG. 9 schematically shows the overall operational diagram and chart including the communication scheme dedicated to sensing swarm data gathering and consolidation. For regions with low cellular coverage, a private cellular network (4G/5G) can be deployed, for instance on a drone. However, the association of spatial and temporal data to measurements requires a form of collaboration between the readers, and in the hierarchical architecture, the collaboration is enforced by the gateway-cloud node. But this node does not need to be a physical device, it can be virtual.

In 5G, most network functions are virtualized, and specific edge functions can be deployed near the actual execution location to minimize latency. The virtual gateway cloud generates the time signal to synchronize all readers and runs more advanced functions: data cleaning, localization, spectrum optimization. The latter is understood to minimize spectrum usage by the reader, for instance by leveraging the sensors' redundancy.

### Constrained Environmental Sensing With SDP Swarms-Related and State-Machine Operation

Undoubtedly, real-world trials are the best choice for validating the performance and efficacy of the SDPs of the present invention. However, at this stage of the invention, real-world trials would introduce intrinsic and extrinsic confounding factors that are hard to control, and, as such, might introduce errors to the analysis process at this stage of research. Therefore, ‘artificial’ mixtures of chemical compounds that simulate the application of interest will were used instead to precisely determine the chemical nature, sensitivity, and specificity of each individual chemical compound in the targeted mixture as well as the necessary iterative feedback during SDP optimization without the intervention of (disruptive) parameters, such as uncontrolled contaminations, fluctuations in temperature and humidity, etc. This analysis was benchmarked with standard, lab-based mass spectrometry. Further complimentary analysis includes estimation of the mechanical forces on each individual or ensemble of SDPs and in-flight analysis.

### Sensing Gas Mixtures with the SDP Devices

Reference is made to FIG. 6A schematically showing the experimental arrangement for the SDP devices (100) for the gas (VOC) mixture (101) sensing inside the simulation chamber (102) using multiband and single frequency measurements scheme when the single SDP units are kept at a longer distance. FIG. 6B demonstrates the experimental arrangement for the SDP devices for the gas (VOC) mixture (101) sensing inside the simulation chamber using multiband and single frequency measurements scheme when the multiple SDP units are placed very close to each other, and the concentration mapping during the elution of the mixture from the simulation chamber. FIG. 6C schematically shows the principle of the operation of the SDP devices, where an RF source represents a spherical waveform reaching all the SDPs simultaneously. Consequently, a frequency tuning as shown in FIG. 6D and explained below must be done to take the data wirelessly.

Optimized SDPs are randomly distributed in simulated environment within a specially-designed tubular gas chamber as shown in FIGS. 6A and 6B. The dynamics and positional arrangement of the SDPs were modelled in various flow conditions (0.1-20 cm³ per minute, standard cubic centimetre per minute), laminar and turbulent flow modes, and various mixtures containing 5-100 various toxic volatile organic compounds (VOCs). The specific ratio of examined and background VOCs (e.g., 2:1, 5:1, 10:1) was chosen as per maximum toxicity limiting value (TLV) to normal background VOCs as set by EPA. While in chamber, the SDPs were scanned with an external RF power source and data acquisition system collected the spectrum of signals emitted from each SDP with relation to the distance from VOC(s) injection source. This process was examined as a function of time, and a multi-layer dynamic VOCs map was obtained using similar algorithms to those mentioned above.

Considering the random distribution of devices scattered in every place, there is two possibilities could occur, namely as, close distribution and wide distribution. For close distribution, the multiple SDP devices are kept very near to each other and multiband frequency measurements are performed to selectively scan the devices (FIGS. 6B-6C) with their designed distinct resonance frequencies (f₁ . . . f₅).

For the wide distribution, single SDP device is kept at large distance (FIG. 6A), and one specific frequency is used to scan the device. For both multi band/single frequency mode, external scanner was moved along the simulation chamber wall to map the spectrum as a function of time and position as shown in the 2D graphical representation in these figures.

By the end of each experiment, biodegradability study was performed by following standard ISO protocol. The SDP devices are considered readily biodegradable if 60% (or 70% for some tests) of the organic carbon in the material is converted to CO₂ within a 10-day window and within 28 days total (ISO 9439: aerobic biodegradability of organic compounds in an aqueous medium). The SDP samples were kept in a vial with aqueous medium and soil sample.

The VOC headspace was extracted and evaluated to monitor the degradation kinetics. Other SDP's metallic components (e.g., Zn or Mg) of the printed electrodes/antenna were readily dissolved in water or simulated body fluids (Hanks' solution; pH 5-8) and dissolved solution could be monitored by UV-vis spectroscopy or inductive coupled plasma (ICP) MS spectrometry to quantify the presence of metallic samples in water. Of notion, the biodegradable polymeric coating (polylactic acid (PLA) and polyglycolic acid (PGA), polycaprolactone (PCL), silk fibroin) across the surface of SDP device could be dissolved in water due to dissolution and the swelling of the polymers for water. The degradation kinetics was monitored by time dependent FTIR for identifying dissolved biochemical ligand from solution in regular intervals.

### Quality Control of SDPs During Aerosolization

In real applications, discriminating good from bad SDPs in the case of mechanical thrust, which may cause malfunction in electronics inside, is of special importance. This could lead to a defected circuit components that will significantly vary from the designed impedimetric network and, thus, shift the resonance frequency. For this reason, the quality and related stability of the SDP components when aerosolized in the environment are examined.

Reference is made to FIG. 6D showing a frequency tuning, which must be done to take the data wirelessly. After the fabrication and dicing of mass scale printed devices as mentioned above, the SDP devices were serially kept inside the testing chamber with specific RF excitation from outside. The scanner continuously tuned the RF power of each device and monitor the resonance frequency of the impedimetric network (sensor+antenna). Any deviation from designed ideal frequency of good device (f_{G}) were considered as mechanical malfunction or defect (f_{D}) and the related SDP was sorted out (see FIG. 6D). During the testing in real environments, the intelligent monitoring system continuously interrogated about the quality of the devices.

### In-Flight Analysis

This analysis helped tracking SDPs in a real environment of land/atmosphere via a drone or station. This was achieved by tracking the spectrum of signals acquired from floated or precipitated SDPs and correlating them with their speed, air and SDP densities, dimensions and particle drag coefficient. A statistical approach was implemented for organizing the distributed spectrum in each case (flight and precipitated mode). In addition, a swarm algorithm was implemented for simulating very large number of injected devices from their communicating behaviour. This is important because the number of SDPs in real scenario could be billions and, therefore, a computationally efficient algorithm based on nature will be adopted to minimize the large channel capacity requirements.

### Benchmarking with Standard Gas Chromatography

Benchmarking SDP performance with laboratory-based gas chromatography for various simulation and real samples was carried out. Mixed chemical compounds, such as those mentioned above, were analysed with standard GC protocol. This result was standardized by inspecting retention time and relative abundance of specific chemical compounds in conjunction with statistical analyses. After identifying the targeted chemical compound and respective time position, the results were compared with HSGC-and SDP-derived chromatogram to know about the specific time interval where the maximum variance could be found.

### Real-Time In-Situ Detection and Mapping of Compounds Using Artificial Intelligence

In some embodiments of the present invention, machine-learning methods are used for detection and identification of chemicals from multiple sensor signals received and processed. Since time dimension needs to be considered, specific network architectures based on recurrent neural networks or long short-term memory blocks were investigated. The challenge in these models is not only the variability and amount of input signals, but also the ability to deploy model instances at multiple nodes and to execute them within real time constraints.

As an example in the present invention, the following were used in the machine-learning methods: (i) dataset of 100-1000 multi-sensor time series-signals; (ii) databank with curated and annotated multi-layer, multi-sensor signals (more than 20 TB of signals); and (iii) machine learning model for target compound detection in real time (accuracy in single-sensor signals is more than 75%, and multi-sensor signals-more than 70%); and time to obtain results was less than 30 sec.

### Machine Learning and Real-Time Analysis

In the present invention, the best suited machine-learning architectures to analyse multiple sensor signals considering the time domain are designed. A supervised approach is followed during the development stage to train the models by using consolidated and contextualized data from the output of the VOC analysis. The first step consists in generating the dataset for training the models. This requires annotating the signals by identifying target compounds, as well as dealing with the organization of the data to consider signals from the multiple layers and the time dimension. The best approach to pre-process and clean the signals to use them to train the network was investigated. Afterwards, a neural network based on architectures proposed in the literature was designed and adapted to the problem to be solved in the present invention.

The generated machine-learning model may include any of the following algorithms: a support vector machine (SVM) based process; a decision tree-based process; and a deep neural network (NN) process, wherein the deep neural network is one or more models of a convolutional neural network (CNN), a regional CNN (RCNN), and a long-short term memory recurrent CNN (LSTM Recurrent CNN).

To deal with the time domain, a recurrent network, such as LSTM Recurrent CNN, is used in the present invention, including the specific blocks, layers, and connections within the network architecture, as well as the best loss function. Hyperparameters were optimized after designing the model. Finally, the models were reduced to deploy them in the edge nodes that read the signals for their execution in real time (edge computing). The models were generated using the tensor flow framework, so we the tensor flow Lite was used for the reduction.

### Tumour Diagnostics with the Wearable Molecular Imaging-Based Tomography Devices

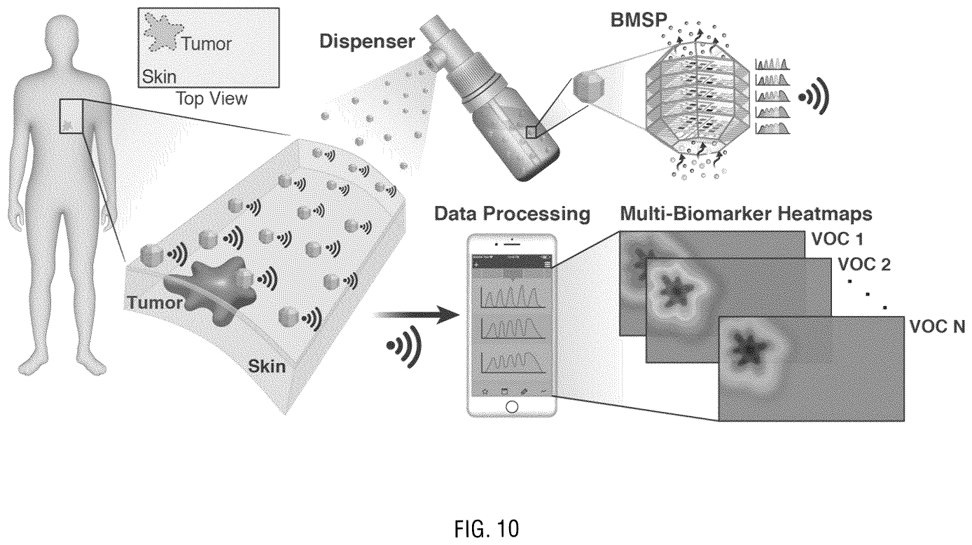

In a further embodiment, wearable molecular imaging-based tomography devices of the present invention are used for lifelong non-invasive and continuous monitoring and molecular imaging of breast cancer. This is achieved by SDP swarms-related networks that carry out spatiotemporal targeted and non-targeted identification of volatile organic compounds (VOCs) linked with the disease as shown in FIG. 10. The figure shows a design concept of the method of the present invention for tumour diagnostics. A dispenser applies thousands of submillimetre, RF-enabled biocompatible SDPs as a thin film on the skin overlying tissues of interest.

Spatiotemporal electrical information is generated by the SDPs upon interaction with a wide spectrum of volatile organic compounds (VOCs) released from the skin. This data is transmitted to a signal receiver and processed by Al. The output is a heatmap (chemical tomogram). The identified VOCs and points of interest can be viewed by zooming in and out of the map in real-time. The SDP devices are biocompatible and do not harm the skin, human body or environment.

To achieve continuous and unobtrusive monitoring of health status and early detection of cancer development, hundreds or thousands of SDPs must be applied as a long-lasting spray to the breast's skin in a manner that accounts for the topology of the breast as a three-dimensional object.

Each SDP includes ground-breaking arrays of magnetoelectric (ME) antennas integrated with a graphene nanocavity (GNC) with a unique identification (ID) barcode so that each individual SDP can be detected and assigned a unique code in the Al system as well as with (X, Y and Z) coordination on the breast. The combined outcome from all sprayed SDPs is a spatiotemporal map (chemical tomogram) in which each pixel represents a wide spectrum of targeted and non-targeted VOCs that are produced by the underlying tissues. Therefore, the sprayed SDPs enable the sensitive and specific detection of premalignant and early-stage lesions of breast cancer on one hand and different molecular subtypes of breast cancer on the other.

As described in the present invention, the overall design principle of the SDP relies on a time-space-resolved geometry with a (porous) cellulose-based membrane that induces time lags of various compounds found in a complex mixture, similar to conventional gas chromatography (GC). This is combined with an array of sensors that detect each eluted compound by an Al-empowered fingerprint of electrical/digital signals, similar to mass spectroscopy (see FIG. 10). Once on the skin, each SDP sends a uniquely encoded series of VOC-specific spatiotemporal signals in response to applied electromagnetic signals, using a wireless reader that operates with radio frequency (RF) backscatter. Machine learning models that collect all spatiotemporal signals from every SDP, whose unique fabrication ID are linked to its coordination on the skin surface, construct a real-time in situ 2D spatial map for each detected VOC, as shown in the figure.

The combined 2D spatial maps of all VOCs form a computerised biomarker-based tomography map of the area. In this map, each pixel contains data about the levels of a wide spectrum of targeted and non-targeted VOCs. The readout from the sampled SDPs will be used to create tomographic molecular (VOC) images of the lesions beneath the skin in a highly sensitive and specific manner. Being able to detect, identify and map biomarkers (VOCs) with high spatial resolution anywhere in the human organ/body in a wearable, non-invasive and continuous manner is an enormous challenge for currently available technologies. Therefore, the successful realisation of the present invention represents a ground-breaking high gain that definitely goes beyond the current scientific and technological knowledge in continuous health monitoring, (bio)sensing and spectrometric technologies, molecular imaging, and wireless communication approaches as described above. It has a very broad impact in a range of medical and biomedical fields.

Applications of the present invention include, but are not limited to, affordable personalised detection and characterisation of the earliest stages of disease development through network-based identification and validation of multiple interacting biomarkers. This is achieved without the need for a complex and expensive clinical infrastructure or for invasive biopsies, proactively providing diagnosis and guidance for practitioners.

In a certain embodiment of the present invention, electrochemical tomograms not only achieve ultra-sensitive network-based identification of diseases, such as breast cancer, but also analyse biomarkers as annotated molecular networks (AMN) rather than as discrete, disease-related entities. This is essentially capitalised upon by the present technology to offer personalised continuous monitoring and surveillance in a manner that would not be possible through the simple assessment of a limited profile of biomarkers that are linked with a specific disease.

Continuous surveillance of individuals with a high risk of developing breast cancer means that early tumours can be detected and treated immediately after their initiation, circumventing the need for disfiguring preventive mastectomies. The success of therapies, for example, in the neoadjuvant setting, can be monitored continuously in real time, facilitating personalised patient-specific therapy regimes that optimise the efficacy of the treatment.

Post-treatment follow-up of breast cancer survivors using the SDPs spray of the present invention for continuous surveillance allows very early recurrence to be detected and localised, facilitating effective and timely intervention for recurrent disease, even many years after primary treatment. This approach rides on breakthrough findings from the present inventors, showing that miniaturised spectrometers based on stacked layers of cellulose and graphene sensors could detect, classify and continuously monitor a wide spectrum of VOCs from complex mixtures, including those emitted from breast cancer tissues.

The long-term applications of the present approach and technology are diverse, paving the way for a plethora of breakthrough innovation opportunities, in addition to those foreseen for breast cancer surveillance.

Thus, one of the embodiments of the present invention is a method for tumour diagnostics comprising applying the SDPs of the present invention as a thin film onto skin of a patient or to a skin ex vivo sample taken from the patient, transmitting spectrometry data received from said SDPs to a signal receiver, processing said data by Al and performing an autonomous analysis to output the result as a heatmap defined as a chemical tomogram as shown in FIG. 10.

The aforementioned SDPs used in the method of the invention are applied to the patient's skin in a form of a spray above premalignant and tumorous tissues. Signals from said SDPs applied to the patient's skin are sampled and analysed through the RFID out-input tags. Biomarkers identified by said SDPs are detected through the patient's skin. Said AI analysis is performed to define biomarker combinations.

The diagnosed tumours and biomarkers are from different types of cancer. Non-limiting examples are pre-malignant breast lesions, breast cancer cells and tumour microenvironments, cultured human cell lines from premalignant breast lesions, such as 21PT, which is atypical ductal hyperplasia and 21NT cells, which is ductal carcinoma, breast cancer cells that represent the different molecular subtypes of breast cancer, such as MCF-7,T47D (luminal), BT474, SKBR3 (HER2), MDA-MB-231, BT549 (triple negative), CTC-ITB-01 (luminal), endocrine therapy resistant, and stroma, such as fibroblasts, extracellular matrix, immune cells, and vasculature.

In another embodiment of the present invention, the SDPs and the system of the present invention are used in tomography and tumour diagnostics.

### EXAMPLES

### Non-Invasive Decoding of Cancer Organoids with The SDPs of The Present Invention

Organoids have captured the attention of the scientific community as a potential revolutionary in vitro model for human organs. These small structures have the ability to mimic organ function and disease, providing a wealth of insights into development, treatments, and disease. However, organoid research faces a major challenge in developing non-destructive methods for monitoring and predicting their growth and behaviour. Prior art methods, such as live cell imaging and genetic analysis, have been instrumental in advancing the understanding of organoid biology and cellular dynamics. By integrating multiple omics approaches, cellular behaviour can be linked with molecular data to aid in the discovery of biomarkers and the development of therapies. However, the limitations of these techniques can impede research progress and delay the clinical applications of organoids.

Conventional laboratory techniques, such as Western Blot, ELISA, proteomics, flow cytometry, PCR and RNA sequencing, pose significant obstacles in organoid studies. They provide functional insights, specificity, sensitivity, protein interaction elucidation, and quantitative data. However, when applied to real-time, in-situ monitoring of organoids, these methods face limitations, including destructive procedures, high costs, low throughput, inability to capture dynamic transition. Specialized skills and extended processing times further restrict their suitability for extensive multi-omics research and clinical applications.

Real-time imaging techniques such as histochemistry, immunostaining, and confocal microscopy provide intricate visual data that is essential for in-situ study of organoids. They offer detailed resolution, enabling precise analysis of organoid structures and functions. However, the use of fluorescent reporters in these techniques can cause phototoxic effects and sample motion issues. Fixation and staining can alter the natural cellular microenvironment and introduce artifacts.

Light sheet imaging is another valuable method, but it has its own set of limitations. It is labour-intensive, potentially destructive to samples, and lacks automation. Moreover, its temporal resolution is limited and struggles with light penetration in denser samples. In addition, integrating complex optical systems into incubators can cause environmental instability, adversely affecting organoid development. This setup also only provides intermittent data, failing to capture the continuous changes in organoid development.

In a further aspect of the present invention, a portable, non-invasive and non-destructive testing approach is introduced that aims to address the invasive and destructive nature of traditional organoid characterization methods. The approach is centred around volatile organic compounds (VOCs), which are crucial signalling molecules that carry important physiological and developmental information. These molecules are emitted into the gas environment around the organoid, known as the headspace.

The SDPs of the present invention and an AI-powered image processing generative deep neural network inspired by insect vision allow to detect and monitor the spatiotemporal distribution of these VOCs. As such, the monitored VOCs provide a digital reflection of the various parts and molecular, genetic and proteomic components of the organoid and has the potential to predict organoid progression from normal to diseased states. This clearly paves the way for a new frontier in organoid research without revealing specific outcomes.

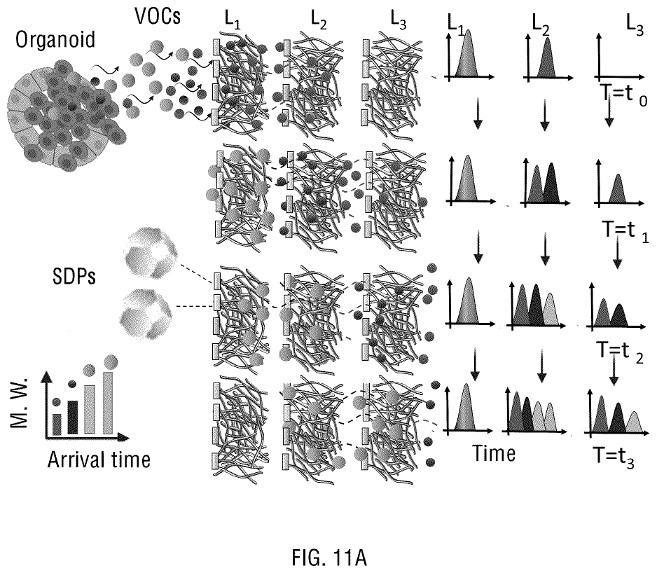

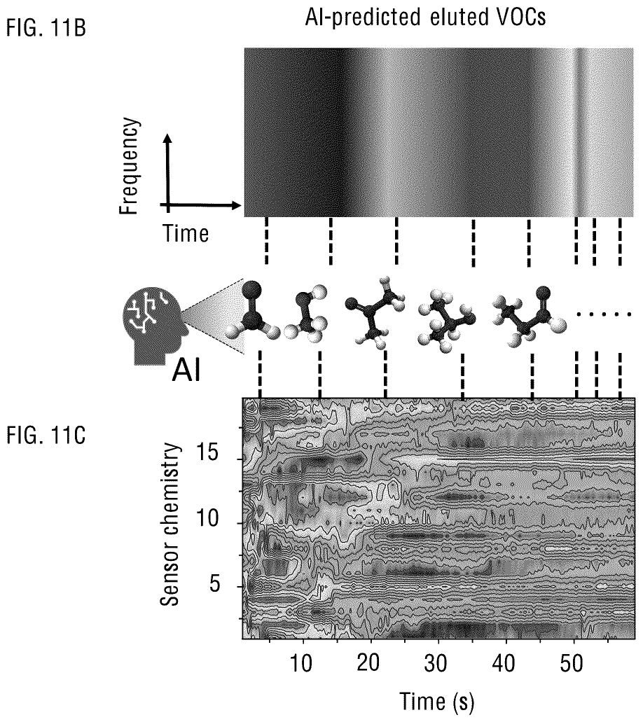

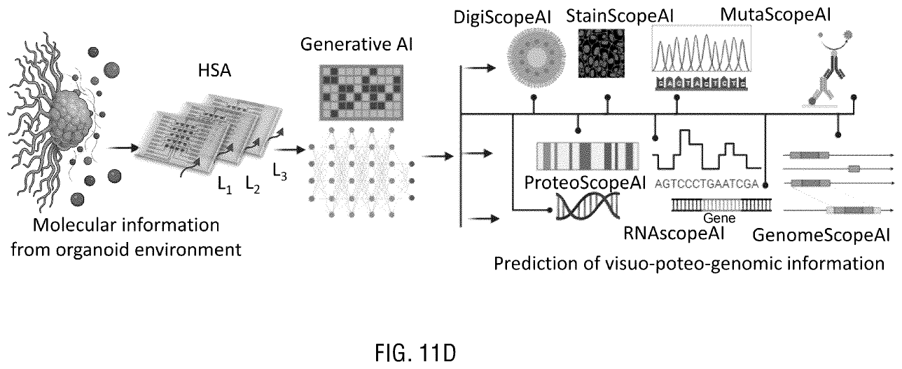

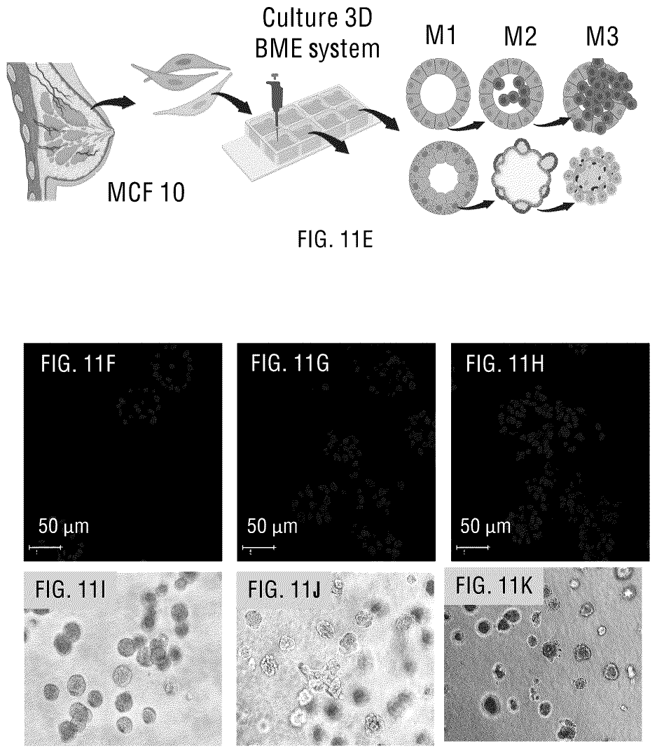

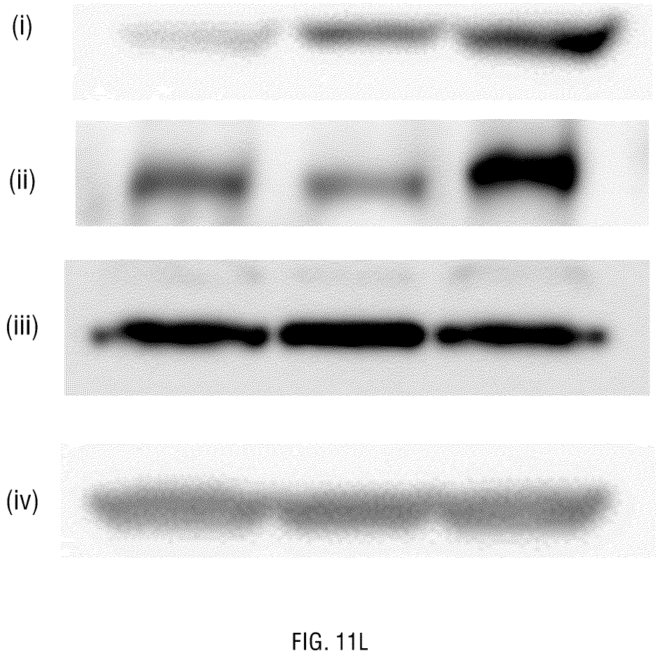

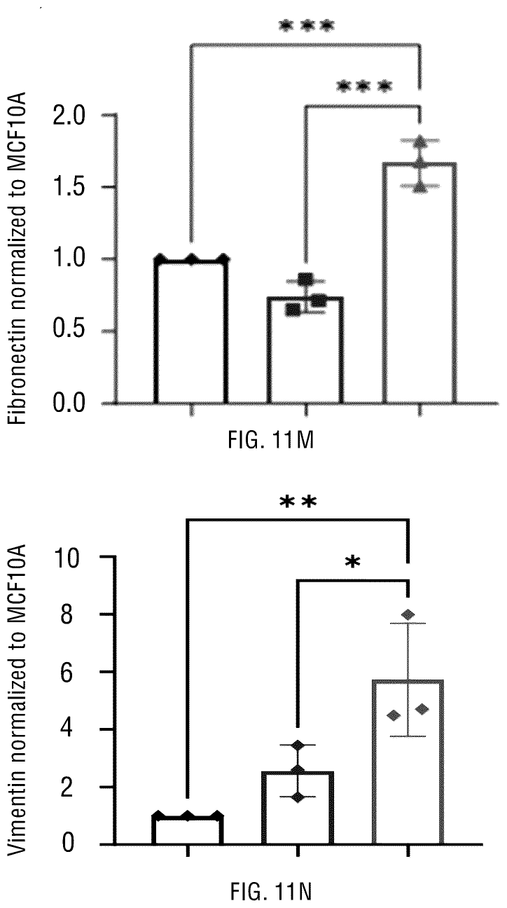

Reference is now made to FIGS. 11A-11N showing the bridging multi-omics data with AI enabled SDP device. FIG. 11A shows the AI-enabled SDP devices of the present invention that elucidate the spatiotemporal separation of VOCs based on molecular weight from the organoid samples' environment. FIG. 11B shows the SDPs map of various eluted volatile organic compounds (VOCs) from the organoid using frequency domain spectrogram, and FIG. 11C shows the 2D chemical tomography through sensor fusion. FIG. 11D schematic represents the bridging volatolomics, which is the study of VOCs emitted by a biological system, under specific experimental conditions, with multi-dimensional imaging as well as proteo-genomics using generative AI.

FIG. 11E shows a stepwise scheme demonstrating breast cancer progression (normal (MCF10A (M1)), premalignant (MCF10AT (M2)) and malignant (MCF10CA1h (M3)) breast cells) and its characterization with DAPI staining shown in FIGS. 11F-11G.

FIGS. 11I-11K shows the microscope images (with magnification ×40, Bar=50 μm), and FIG. 11L shows the western blot (WB) for the expression of mesenchymal markers (i) fibronectin, (ii) vimentin), (iii) E-cadherin (epithelial marker) with a (iv) simple protein loading (tubulin). FIG. 11M and FIG. 11N shows the quantification of fibronectin and vimentin levels, respectively, from WB results. Densitometry values were normalized to M1. Columns; mean, bars; SD, n=3. * P<0.05, ** P<0.01, *** P<0.001.

The SDPs used in the system shown in FIG. 11A induce time lags and distinguish compounds in complex mixtures with time-space resolution. By utilizing AI analysis to generate individual spatiotemporal electrical/digital signals for each eluted compound, it is possible to non-invasively, continuously detect, identify, and map VOCs in specific environments.

When VOC signals are deployed as a network in a specific area/environment of interest, real-time 2D spatial maps can be created by integrating machine learning models for each detected VOC, resulting in a comprehensive computerized chemical tomography map of the analysed area, as shown in FIG. 11C. This map is comprised of all accumulated 2D maps of each VOC, with each bright spot representing the full spectrum of VOCs present at that specific location (se FIG. 11B and FIG. 11C).

Not only the SDP of the present invention offers an ultra-sensitive identification of chemical markers, but it also provides unprecedented continuous monitoring and surveillance beyond assessing a limited profile of disorder-linked VOCs. Continuous monitoring of high-risk areas enables immediate detection and response to anomalies, reducing the need for intensive preventive measures. It also allows real-time assessment of chemical treatments, ensuring optimized, environment-specific regimens.

The conceptual framework for evaluating organoids based on the use of the SDPs involves utilizing different generative-AI models. FIG. 11D provides a schematic representation of these framework, which includes bridging volatilomic information (information on VOCs) to physical surface texture (microscopic and staining image) and generating proteo-genomic information through various AI architectures.

The AI-Powered SDP were exposed to the headspace of breast organoids depicting the progression from a single cell to a malignant stage using a 3D BME system (see FIG. 11E). It begins with MCF 10A (M1) cells forming a healthy breast organoid with a circular acinus structure, advancing through a premalignant stage (MCF 10AT (M2)) to a fully malignant stage (MCF10CA1h (M3)). Microscope and DAPI-stained images (see 11F-11K) show this transition, evidenced by changes from a hollow to a filled lumen and an increase in cell density.

These figures evidenced the morphological changes and surface characteristics. Western Blot analysis (FIG. 11L) clearly indicates that this progression involves Epithelial-to-Mesenchymal Transition (EMT), marked by increased expression of mesenchymal markers, such as fibronectin and vimentin, with partial changes in epithelial markers, such as, E-cadherin, (iii) in FIG. 11L. These findings, including control results with tubulin, (iv) in FIG. 11L, are summarized in FIG. 11L demonstrating the molecular shifts during the transition from normal to malignant states.

### HSA-Based Profiling from Organoid Environment

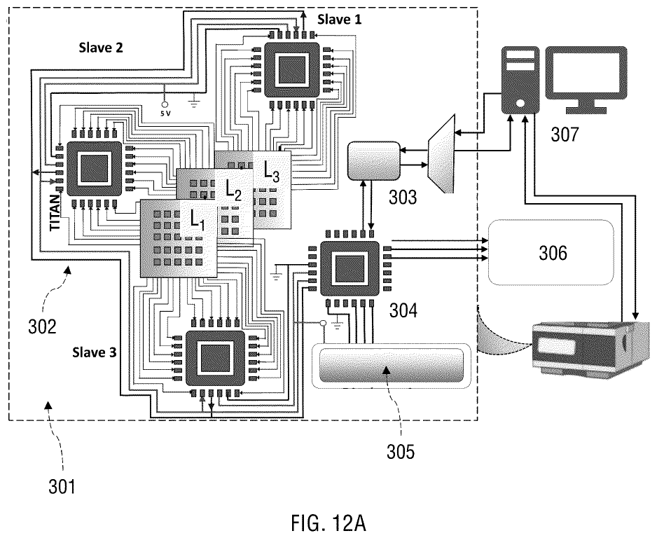

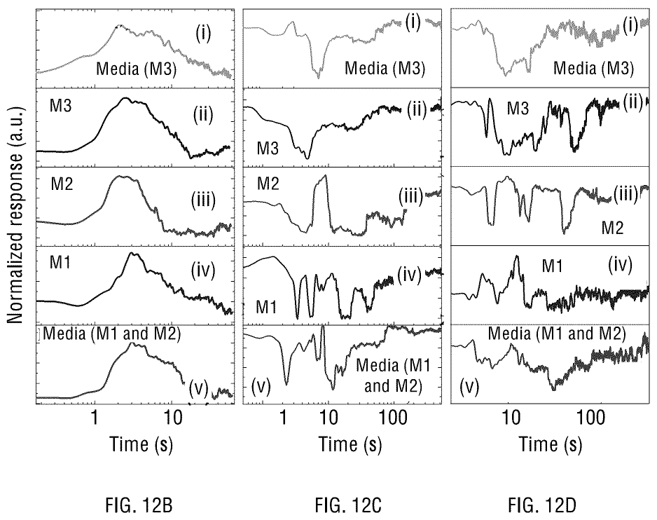

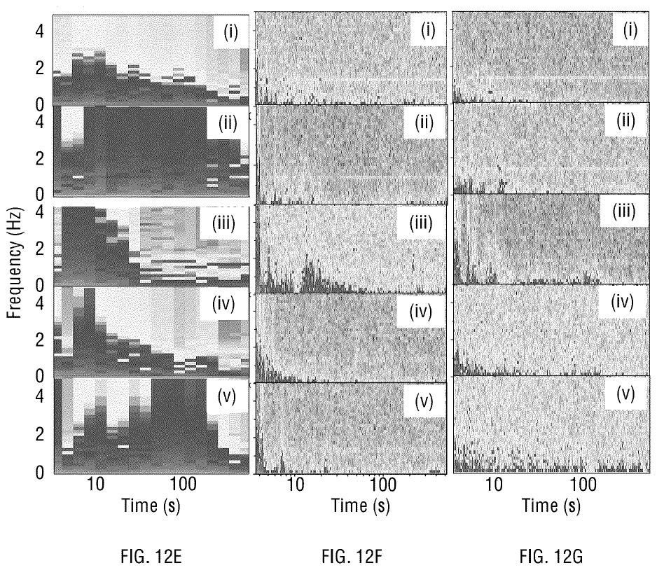

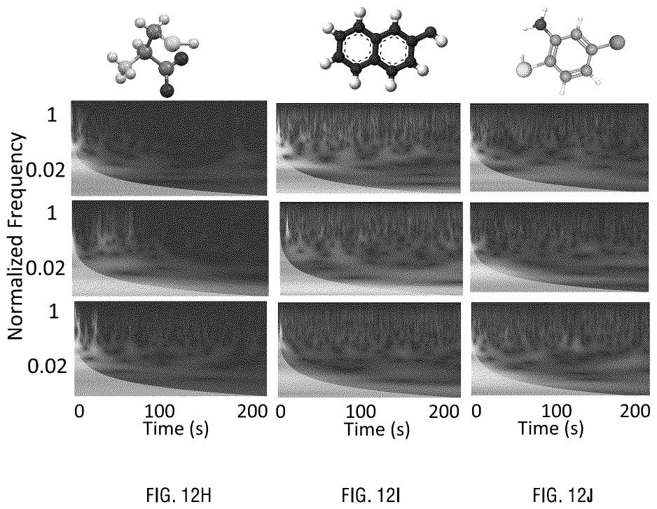

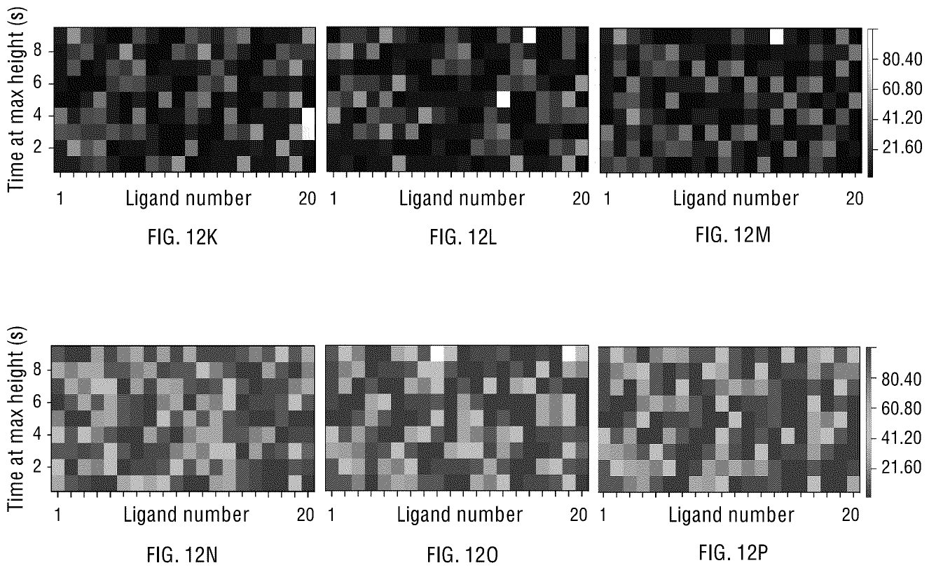

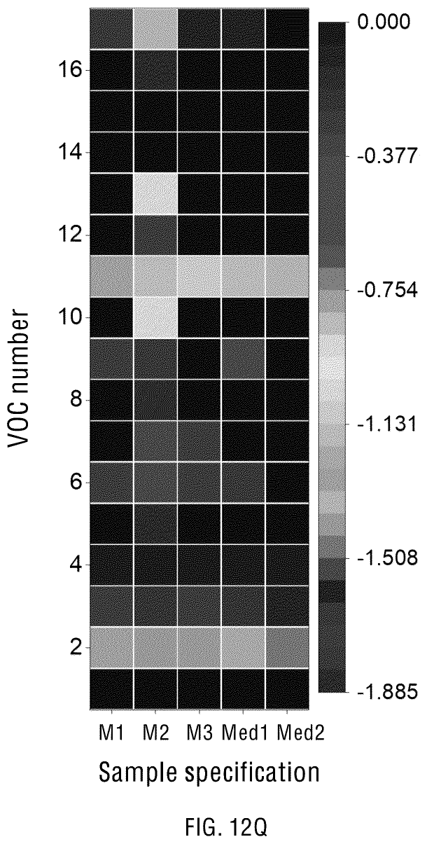

Reference is made to FIG. 12A showing the spectrometer circuit module (301) used for extracting VOCs from organoids with the SDP of the present invention. The circuit and block diagram of the spectrometer module to interface the SDP's layers L1, L2 and L3, as seen in the figure, with I²C communication (302) between Slaves 1, 2 and 3, communication port (303), Master (304), display unit (305), temperature, flow, switching and other controllers (306) and data acquisition and storage system (307). The convergence of the SDP's layers is achieved through the meticulous connection of voltage lines to each sensor within the architecture (see FIG. 12A) enclosed with an inlet on the top layer and an outlet on the back of the third layer.

FIGS. 12B-12D shows the layer-dependent sensing using the SDP of the present invention and their comparative spectrum analyses for M1-M3 and the respective background (media) for Layer 1 (FIG. 12B), Layer 2 (FIG. 12C) and Layer 3 (FIG. 12D) using 1-4-phenylene-diamine-based sensing probe.

Thus, FIGS. 12B-12D present the layer-dependent sensing profiles for all sample (M1-M3) types compared to their respective background media. These profiles are demonstrated for a specific type of ligand, such as 1,4-phenylenediamine. In FIG. 12B, the top layer generates a traditional Gaussian-like sensing profile, while the sensing profiles for each case undergo substantial alteration, displaying split sensing profiles marked by multiple peaks (FIG. 12C). Each sensing pattern remarkably diverges from one another and notably varies from their corresponding background media. As seen in Layer 3 (FIG. 12D), the number of peaks surpasses those observed in Layer 2, indicating enhanced separation capabilities.

In conjunction with the sensing profiles, the corresponding spectrograms are presented in FIGS. 12E-12G for Layers 1-3, respectively, highlighting frequency mapping where specific band formations at distinct times demonstrate the unique presence of specific VOCs or their combinations. This robust separation and identification strategy serves as a potent non-destructive testing (NDT) method for organoid identification based on the distinctive molecular presence. In Layer 1, the spectrogram displays a broad and continuous spectrum, whereas in Layers 2 and 3, the spectral data reveal the specific appearance of bands within certain time frames.

FIGS. 12K-12P shows the heatmaps from gaussian fitting of splitting sensing profile in Layer 2 (FIGS. 12K-12M) and Layer 3 (FIGS. 12N-12P) for M1-M3, respectively, measured with 20 different ligands (X axis) for each case. The heat map generated by the GC-MS analysis for each of 36 VOC samples (VOC1-VOC36) is presented in FIG. 12Q, vividly illustrating the qualitative and quantitative shifts in molecular presence based on their vertical position. The values on the right-hand side of the scale are represented in logarithmic units.

### Synthesis of Distal Patterns of Organoid Images Using Digiscope-AI and Stainscope-AI

Based on the spatiotemporal signals of the various organoid progression stages, an encoder-decoder architecture known as U-Net was used to generate microscopic and DAPI-stained images. This architecture translates one image to another image, as explained above. Input mosaic images were created by merging sensing profiles from 20 different sensor chemistries within a given layer, simulating synthetic superposition inspired by compound vision observed in insects.

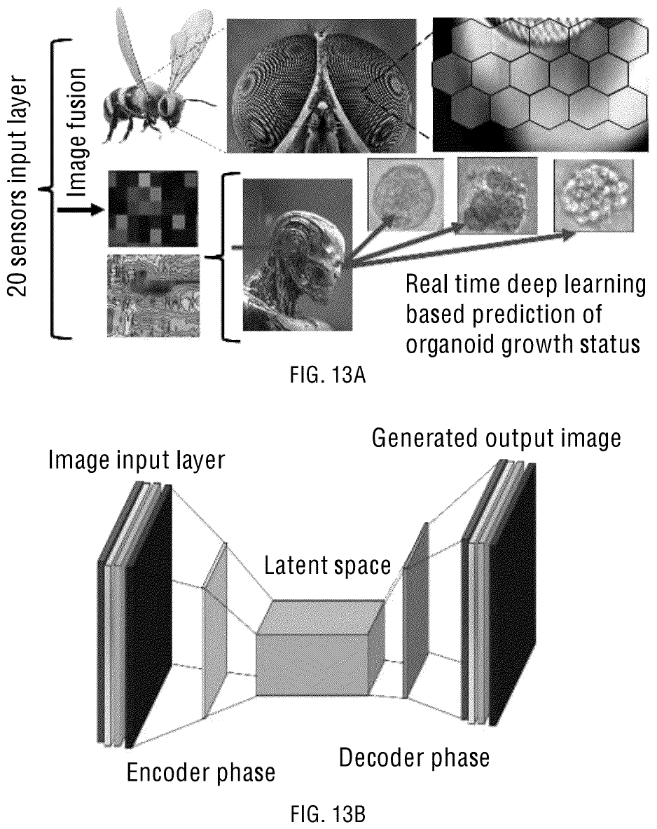

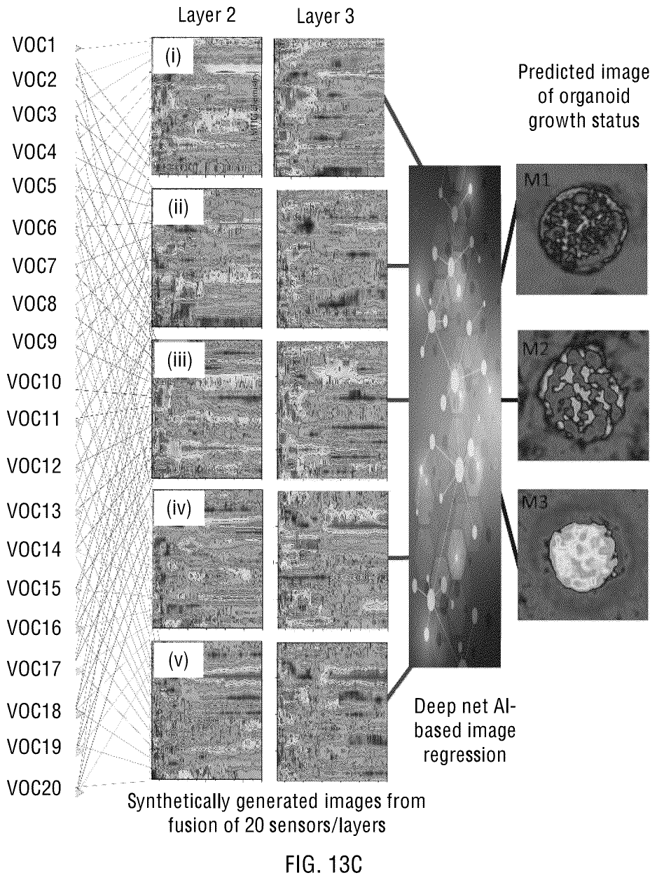

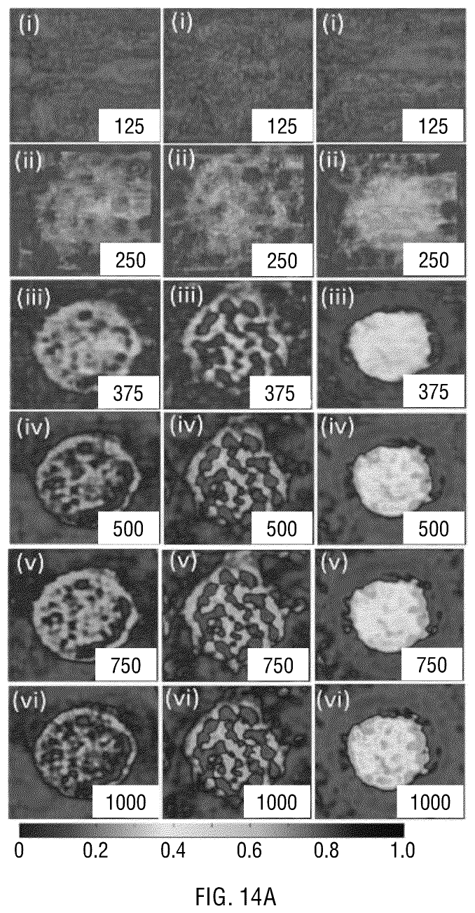

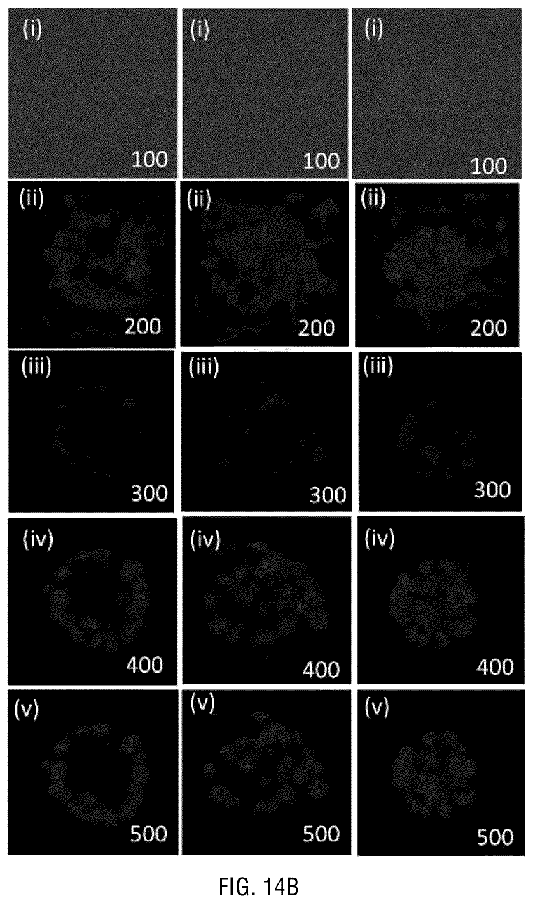

Reference is now made to FIGS. 13A-13C showing the compound image processing inspired from ‘ommatidium’ mechanism in ‘bee vision’ to predict organoid's heterogeneity. FIG. 13A illustrates the concept of the invention which is creating hybrid compound mosaic images (2D tomography) by amalgamating sensor chemistries within distinct layers. These images are then harnessed in deep neural network image processing to predict the specific organoid's image pixel by pixel.

FIG. 13B schematically shows generative deep learning models for organoid image synthesis using an encoder-decoder based U-net architectures tailored for generating organoid images, both stained and unstained, as detailed further in FIGS. 14A-14B (showing hierarchical steps of the architecture in extended data). FIG. 13C schematically shows the sensing profile from 20 sensors/layers. The utilization of 20 sensor inputs yields synthetic superpositions for each layer (Layer 2 and 3), resulting in distinctive mosaic arrangements. These arrangements not only differentiate M1, M2, and M3, but also set them apart from their respective media backgrounds. This innovative approach holds the potential to serve as a sophisticated alternative to intricate in-situ imaging, effectively monitoring the growth status of targeted organoids.

The SDPs or the SDPs swarm of the present invention combines spatial, temporal, and sensor interdependence data to produce images of microscopic structures, DAPI-stained samples, and proteo-genomic heatmaps. By leveraging compound vision and using U-net encoder-decoder learning architecture, as shown in FIG. 13B, the framework of the present invention is capable of generating distinct mosaic patterns for all samples (M1-M3) and their backgrounds, as seen in FIG. 13C for (i)-(iv). The images generated from M1-M3 are then utilized for deep neural network-based AI image processing using the U-net architecture with an encoder-decoder framework (see FIG. 13B). These images go through multiple training cycles where the AI system learns intricate relationships between input and output pixels.

Reference is now made to FIGS. 14A-14B showing the generative deep learning models for organoid image synthesis. The resulting AI-generated images for different numbers of training cycles demonstrate regular microscope images as seen in FIG. 14A. Initially, they show normal grayscale images transformed into false colour representations based on the brightness and darkness of organoid surface texture. Each image's training cycle number is indicated in the right-hand side corner at the bottom of the image.

FIG. 14B shows the resulting DAPI-stained images for M1, M2, and M3 for different numbers of training cycles. The DAPI images capture staining details through a black-blue hue.

Thus, based on these results, it is clear that the Al system demonstrates its ability to learn the intricate relationships between input and output pixels. As the training cycles progress, the Al system improves image attributes such as texture, brightness, accuracy, count of polarized cells, geometry, and shape, and indicates a higher cell compilation, which is a hallmark of malignant traits. By combining the SDPs or the SDPs swarm of the present invention and Al-driven processing, the present invention allows to generate images with enhanced accuracy and detail, offering practitioners new insights into the structures and functions of microscopic organisms.

### Correlating and Predicting the Proteo-Genomic-Mutation Profile from Volatilomic Information

Computational pathology driven with Al integrates computer vision, microscopic imaging, molecular pathology, genomics, and bioinformatics, thereby revolutionizing cancer histopathology analysis for predicting genetic changes, assessing patient survival, proteome-wide missense variant predictions, creating realistic image patches for stain colour normalization and transforming stains in computational pathology. A further step forward, the present inventors used VOC spatiotemporal profiles to express the proteo-genomic from organoids (M1-M3) to explore the connection between genetic variations and protein expressions.

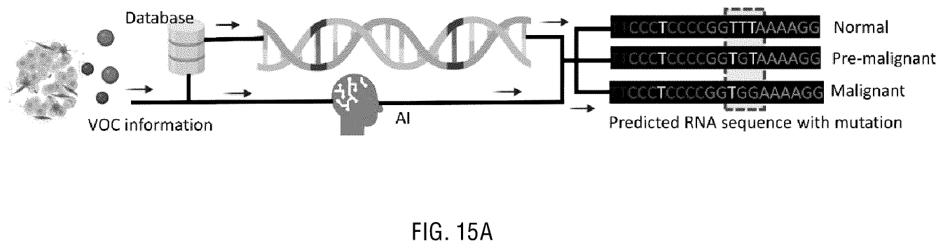

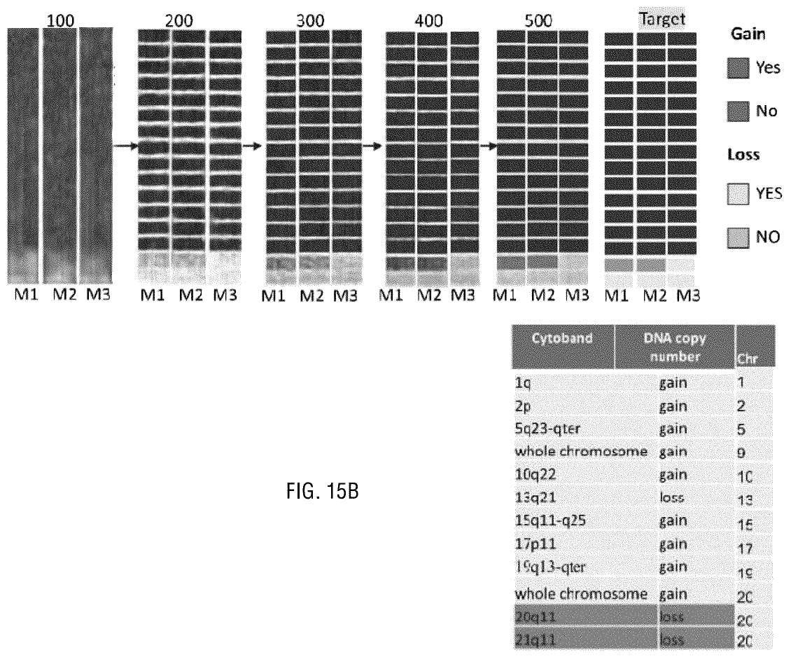

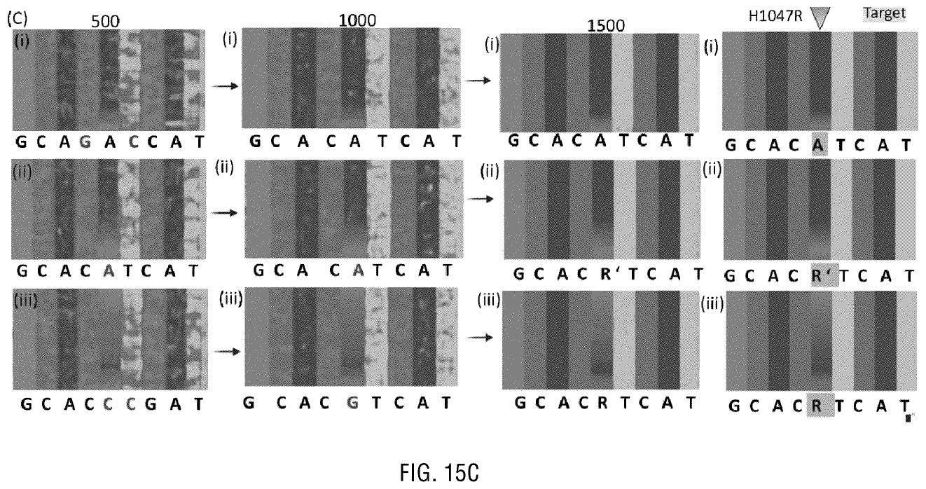

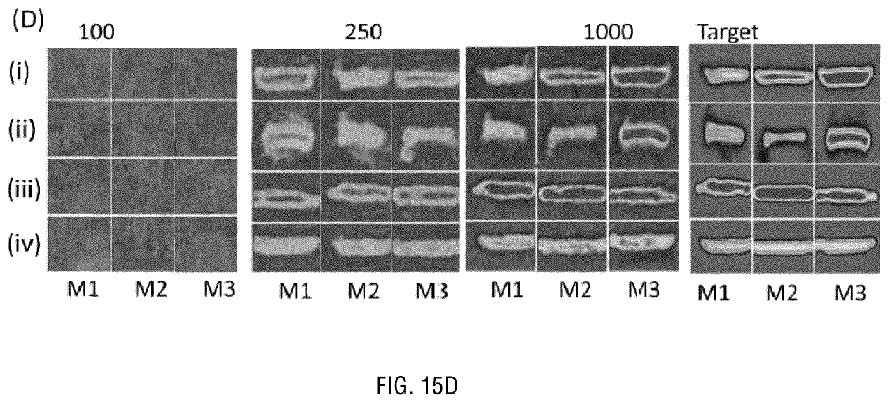

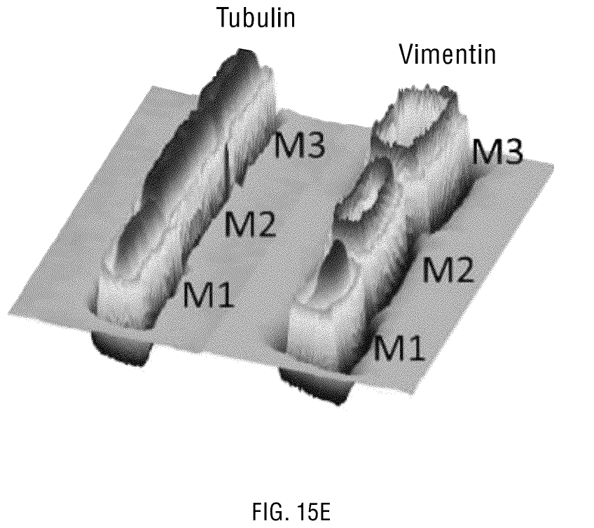

Reference is made to FIGS. 15A-15E showing Genomescope-AI, Mutascope-AI and Proteoscope-AI for volatolomics-proteomics-genomics fusion and genetic prediction of organoids. FIG. 15A schematically shows the AI architecture that explores the VOC-genetic correlation, and FIG. 15B shows the Genomescope-AI predicted DNA copy number variation (CNV) at various cytoband position of target chromosome for M1, M2, M3 in the form of heatmap (Yes/No) for gain and loss. Each case is depicted with distinct colour and the number of iterations with corresponding predicted heatmap output is shown in the top of each case.

FIG. 15C shows the Sequencescope-Al predicted RNA sequences of (i)-(iii) M1-M3 and their mutation position, and FIG. 15D shows the Proteoscope-AI predicted WB results for various protein expressions, such as (i) vimentin, (ii) fibronectin, (iii) E-Cadherin and (iv) tubulin (loading control) for M1-M3. Here tubulin acts as simple protein loading without noticeable change from M1 to M3. FIG. 15A shows a schematic of this relationship, and FIG. 15B presents the Genomescope-AI generated heatmap profile for genomic study of DNA-copy number variation (CNV) at various cytoband positions of target chromosomes for M1-M3.

CNVs are structural variations in the genome where specific segments of DNA are duplicated or deleted, resulting in altered copy numbers of specific genomic regions. These variations may encompass one or more genes, and all the CNV information was correlated and predicted with colour codes heatmap for gain and loss at various chromosome and cytoband positions. This allows for the direct relationship of VOC-CNV, as established through Genomescope-AI. For the CNV (FIG. 15B), accurate depictions of the target heatmap were achieved at a higher number of iterations (~500), as expected.

FIG. 15C (i)-(iii) shows the Sequencescope-AI predicted RNA sequences from a VOC profile in different iteration stages (500-1500) for M1-M3, respectively, for H1047R activating mutation in the PIK3CA gene in human cancer. M1 cells have wild type A-allele marked by an arrow. M2 also have predominant A-allele with very low intensity from G or others. In contrast, M3 cells exhibit predominance of the mutant G-allele in addition to the wild type A-allele. The mutation results in an amino acid change from His to Arg at 1047 (H1047R).

The chromatogram from M. Kadota et al., Delineating genetic alterations for tumour progression in the MCF10A series of breast cancer cell lines, PloS One, 5(2), e9201 (2010), was used as target RNA (expressed through colours, such as, blue (G), green (C), red (A), yellow (T), and their mixed colour combinations in R and R′ with their highest abundance expressed through specific colour's presence to the RNA-scope-AI architecture. Although errors in the prediction of specific RNA sites (marked in ‘red’) are prominent especially in lower iterations, the network can predict with correct RNA sequence in higher iterations (~1500).

Moreover, the present inventors have also tested the VOC-proteomic relation from western blot (WB) results. FIG. 15D shows the predicted results of WB by Proteoscope-AI to generate Western blot (WB) results for various protein expressions, including (i) vimentin, (ii) fibronectin, (iii) E-Cadherin, and (iv) tubulin (loading control) across M1-M3 in various iteration stage (100-1000).

In FIG. 15E, the 3D representation of predicted WB results of vimentin illustrates the quantitative predictions of protein expression through EMT. This transformative process is reflected in the Proteoscope-AI-predicted WB mapping, where there is a noticeable increase in both the area and height for vimentin and fibronectin. Conversely, minimal changes are observed for E-Cadherin and tubulin (loading control).

The training of genetic data and WB images has been intricately correlated with input VOC profiles specific to M1-M3. Subsequently, these correlations have been transformed into colour scale images, utilizing Al-generated representations to provide a comprehensive understanding of the molecular dynamics during the transition from normal to premalignant and malignant states. Overall, the experimental results described here shed light on the intricate interplay between organism's genetic makeup, protein expression, and its volatilomic signature, potentially leading to novel diagnostic and therapeutic insights.

1-34. (canceled)

35. A system for non-invasive, continuous monitoring of skin or biological tissue beneath the skin of a patient, the system comprising:
(a) a plurality of programmable, battery-less, autonomous, and biodegradable smart dust particles (SDPs), wherein each SDP comprises:
(1) a layered separating medium providing physical support and configured for chromatographic separation of chemical compounds from a sample;
(2) at least one detector comprising an array of sensors printed on layers of said separating medium, and configured to generate signals indicative of the presence or properties of said chemical compounds;
(3) embedded electronics printed on layers of said separating medium, operatively connected to said detector; and
(4) at least one radio-frequency identification (RFID) tag connected to said embedded electronics, configured for remote readout and zero-power operation, and comprising a unique identification (ID) barcode associated with said SDP;
(b) an external signal receiver configured to wirelessly receive signals transmitted from the RFID tags of said plurality of SDPs after said SDPs are applied to the skin overlying the biological tissue; and
(c) a processor operatively connected to said external signal receiver, said processor configured to:
(i) correlate the signals received from each SDP with a spatial coordinate on the skin based, at least in part, on the unique ID barcode of the respective SDP; and
(ii) generate a spatiotemporal chemical tomogram representing a map of detected chemical compounds across the biological tissue based on the spatially correlated signals received from the plurality of SDPs over time.

36. The system of claim 35, further comprising a dispenser configured to apply the plurality of SDPs onto the skin as a spray.

37. The system of claim 35, wherein the biological tissue comprises breast tissue, and the chemical tomogram is indicative of premalignant lesions or breast cancer.

38. The system of claim 35, wherein each SDP further comprises at least one component selected from the group consisting of magnetoelectric (ME) antennas and a graphene nanocavity (GNC), integrated with the RFID tag or embedded electronics.

39. The system of claim 35, wherein the SDPs are sub-millimetre in size.

40. The system of claim 35, wherein the layered separating medium comprises layered cellulose paper or layered nitrocellulose film, optionally in an origami or kirigami form.

41. The system of claim 35, wherein the detector is selected from the group consisting of a micro-gas chromatograph, a miniaturized dispersive optical spectrometer, a fibre-coupled optical spectrometer, a MEMS-based spectrometer, a plasmon-enhanced Raman spectrometer, an on-chip plasmonic spectrometer, a piezoelectric crystal detector, a spin-induced mass spectrometer, and combinations thereof.

42. The system of claim 35, wherein the sensors are selected from the group consisting of thermal conductivity sensors, surface acoustic wave (SAW) sensors, chemiresistor array sensors, chemicapacitive array sensors, and nanocantilever sensors.

43. The system of claim 42, wherein the sensors further comprise at least one chemical or biomolecular layer immobilized thereon, capable of binding or adsorbing said chemical compounds, wherein said chemical compounds comprise volatile organic compounds (VOCs).

44. The system of claim 35, wherein the processor is further configured to perform artificial intelligence (AI) analysis on the spatially correlated signals to generate the chemical tomogram.

45. The system of claim 44, wherein the processor performing an artificial intelligence (AI) analysis is further configured to:
i. analyse temporal changes and spatial patterns within the generated spatiotemporal chemical tomogram over the period of time; and
ii. proactively identify or predict a status or change in status of the biological tissue, including onset, progression, remission, or recurrence of a condition, based on the analysed temporal changes and spatial patterns.

46. The system of claim 35, wherein the processor is further configured to monitor changes in the chemical tomogram over time, indicative of disease progression, remission, or recurrence.

47. The system of claim 35, wherein the external signal receiver is integrated into a wearable device or a mobile device.

48. The system of claim 35, wherein the SDPs are configured for remote powering via RF energy harvesting facilitated by the RFID tag or a dedicated antenna.

49. A method of chemical tomography for non-invasive, continuous monitoring of biological tissue, the method comprising:
I. Applying a plurality of programmable, battery-less, autonomous, and biodegradable smart dust particles (SDPs) onto skin overlying the biological tissue, wherein each SDP comprises a unique identification (ID) barcode and sensors configured to generate signals related to chemical compounds proximate to the SDP;
II. Wirelessly receiving, via an external signal receiver, signals transmitted from said plurality of SDPs over a period of time;
III. Correlating, via a processor, the received signals from each SDP with a spatial coordinate on the skin based, at least in part, on the unique ID barcode of the respective SDP;
IV. Generating, via the processor performing an artificial intelligence (AI) analysis, a spatiotemporal chemical tomogram representing a map of detected chemical compounds across the biological tissue based on the spatially correlated signals; and
V. Monitoring the status of the biological tissue based on the generated chemical tomogram and changes therein over the period of time.

50. The system of claim 49, wherein the processor performing the AI analysis is further configured to:
i. analyse temporal changes and spatial patterns within the generated spatiotemporal chemical tomogram over the period of time; and
ii. proactively identify or predict a status or change in status of the biological tissue, including onset, progression, remission, or recurrence of a condition, based on the analysed temporal changes and spatial patterns.

51. The method of claim 50, wherein performing the AI analysis comprises utilizing a recurrent neural network architecture, optionally comprising long short-term memory (LSTM) blocks, to process the spatially correlated signals over time and analyse the temporal changes and spatial patterns within the chemical tomogram.

52. The method of claim 49, wherein applying the plurality of SDPs comprises spraying the SDPs onto the skin.

53. The method of claim 49, wherein the biological tissue comprises breast tissue, and the method is used for lifelong monitoring for premalignant lesions or breast cancer detection, progression, or recurrence.

54. The method of claim 49, wherein each SDP further comprises at least one component selected from the group consisting of magnetoelectric (ME) antennas and a graphene nanocavity (GNC), and wherein wirelessly receiving signals utilizes said component.

55. The method of claim 49, wherein the chemical compounds comprise volatile organic compounds (VOCs) released from the skin, originating from the underlying biological tissue.

56. The method of claim 49, further comprising wirelessly powering the SDPs via radio frequency (RF) energy harvesting.

57. The method of claim 49, wherein generating the chemical tomogram comprises identifying both targeted and non-targeted chemical compounds represented as pixels on the map, and wherein monitoring comprises analysing changes in the patterns or concentrations of said compounds over time.

58. A wearable monitoring device comprising:
the external signal receiver and the processor of claim 35, integrated within the wearable monitoring device or in wireless communication therewith, wherein the processor is configured to receive the signals from the receiver and generate the spatiotemporal chemical tomogram for display or further transmission, thereby enabling continuous monitoring of the skin or biological tissue beneath the skin via the wearable monitoring device, wherein the external signal receiver is configured to be worn by the patient.
